(19) United States
(12) Patent Application Publication
(10) Pub. No.: US 2025/0049791 A1
(43) Pub. Date: Feb. 13, 2025

(54) Synthesis of MEK7 Inhibitors and methods of Use Thereof

(71) Applicant: Northwestern University, Evanston, IL (US)

(72) Inventors: Karl A. Scheidt, Evanston, IL (US); Dalton Robert Kim, Evanston, IL (US); Meghan Jo Orr, Evanston, IL (US)

(21) Appl. No.: 18/792,715

(22) Filed: Aug. 2, 2024

Related U.S. Application Data
(60) Provisional application No. 63/517,604, filed on Aug. 3, 2023.

(51) Int. Cl. A61K 31/505; A61K 31/506; C07D 239/47; C07D 403/12; C07D 405/12

(52) U.S. Cl. CPC A61K 31/505; A61K 31/506; C07D 239/47; C07D 403/12; C07D 405/12

(57) ABSTRACT

Disclosed herein are 4-phenoxypyrimidine compounds and derivatives thereof for use as inhibitors of mitogen-activated protein kinase 7 (MEK7). The disclosed compounds and pharmaceutical compositions thereof may be used in methods for treating a disease or disorder associated with MEK7 activity, including cell proliferative diseases and disorders associated with MEK7 activity, such as cancer.

### CROSS-REFERENCE TO RELATED APPLICATIONS

The present application claims priority to U.S. Provisional Patent Application No. 63/517,604, filed Aug. 3, 2023. The entire contents of which are hereby incorporated by reference.

### STATEMENT REGARDING FEDERALLY SPONSORED RESEARCH

This invention was made with government support under grant numbers CA228431, DA050445, GM008152, and CA188015 awarded by the National Institutes of Health. The government has certain rights in the invention.

### REFERENCE TO AN ELECTRONIC SEQUENCE LISTING

The contents of the electronic sequence listing (702581.02535.xml; Size: 3,026 bytes; and Date of Creation: Jul. 24, 2024) is herein incorporated by reference in its entirety.

### BACKGROUND

Acute lymphoblastic leukemia (ALL) is a deadly blood cancer and the most common hematological malignancy in pediatric patients under 14 years of age. Despite advancements in treatments and improvement in outcomes, ALL remains the most frequent cause of death from cancer before the age of 20, and relapse continues to be the leading cause of cancer-related mortality in children.

Studies have shown that loss of mitogen-activated protein kinase 7 (i.e. Map2k7 or MEK7) repression and consequent amplification of downstream MAPK signaling contribute to ALL pathology. However, known MEK7 inhibitors still have potency issues, as well as selectivity issues by inhibiting a variety of other kinases in addition to MEK7.

As such, there remains a need to develop selective and potent MEK7 inhibitors for treatment of acute lymphoblastic leukemia (ALL).

### SUMMARY OF THE INVENTION

Disclosed herein are 4-phenoxypyrimidine compounds and derivatives thereof and methods for using the same. One aspect of the invention provides for 4-phenoxypyrimidine compounds and derivatives having a formula I, or a salt or hydrate thereof:

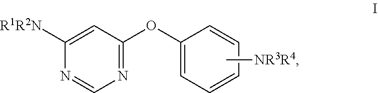

wherein
R¹ is hydrogen or alkyl;
R² is selected from the group consisting of alkyl, cycloalkyl, heterocycloalkyl, -alkylene-heteroaryl, -alkylene-aryl, wherein aryl is optionally substituted with one or more substituents selected from the group consisting of halo, alkyl, haloalkyl, and alkoxy;
or —NR¹R² together form a heterocycloalkyl;
R³ is hydrogen or alkyl;
R⁴ is —C(O)R⁵; and
R⁵ is alkenyl or alkyl optionally substituted with one or more substituents selected from halo and alkoxy.

Another aspect of the invention provides for a pharmaceutical composition. The pharmaceutical composition comprises an effective amount of the compound as described herein, and a pharmaceutical carrier, excipient, or diluent.

Another aspect of the invention provides for a method for treating a disease or disorder associated with mitogen-activated protein kinase 7 (MEK7) activity in a subject in need thereof. The method comprises administering to the subject the compound as described herein or the pharmaceutical composition as described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates the simplified MAPK signaling cascade.

FIG. 2A illustrates the one-pot process towards the synthesis of MAP2K7 inhibitors.

FIG. 2B illustrates in vitro potency of preliminary MAP2K7 inhibitors as determined by the ADP-Glo assay.

FIG. 2C illustrates in vitro potency of MAP2K7 inhibitors with pyrimidine substitution.

FIG. 2D illustrates in vitro potency of benzylamine-derived MAP2K7 inhibitors.

FIG. 3 shows that compound 7 (60 nM) was modeled to bind with its appendant electrophile in proximity to Cys218. However, steric congestion around the electrophilic center leads to less potent ana-logs with substitution nearby (e.g., 10 and 11).

FIG. 4 illustrates the docking comparison of 3,4-methylenedioxyphenethylamine-derived inhibitor (23) and benzylamine-derived 25. Sterics are thought to underlie poor potency of substituted phenethylamine derivatives and better fit of benzylamine analogs.

FIG. 5 illustrates the MEK7/MEK4 selectivity profiles of select inhibitors.

FIG. 6 illustrates the off-target activity profile of 25. Among 90 wildtype kinases surveyed, compound 25 only is appreciably bound by one kinase (EGFR, see dot) at 1 μM.

FIG. 7A shows a summary of active site cysteine residues among the MEK isoforms. Cys218, targeted by DK2403 (25), is highlighted.

FIG. 7B shows that the observed tandem mass spectrometry adduct confirmed DK2403 covalently engages Cys218 (SEQ ID NO: 1).

FIG. 8A illustrates the dose-response cytotoxicity of DK2403 (25) in T-ALL cell lines.

FIG. 8B illustrates the washout cytotoxicity studies of DK2403.

FIG. 8C shows the western blot analysis that demonstrated dose-response (in μM) decreased phosphorylation of JNK and ATF2 in T-ALL cells following treatment with 25.

FIG. 9 illustrates additional selectivity data for compounds 8, 9, and 25.

FIG. 10 illustrates the cytotoxicity profile of compounds 5-9.

FIG. 11 illustrates the detailed cytotoxicity profile of compound 8 (DK133).

FIG. 12 illustrates the PAMPA cell permeability of selected inhibitors. Compounds 5 and 8 were found to be highly membrane permeable.

FIG. 13 illustrates the whole-protein LC/TOF-MS, MAP2K7 and vehicle. MAP2K7 construct was found to correspond to m/z=74310.

FIG. 14 illustrates the whole-protein LC/TOF-MS, MAP2K7 and 25. Complete labeling of the kinase was observed, with some degree of double engagement.

FIG. 15 illustrates the LC-MS/MS of trypsin/chymotrypsin digest and evidence of covalent engagement of 25 with Cys218 (see FIG. 7B) (SEQ ID NO: 1).

FIG. 16 illustrates the LC-MS/MS of gluC digest. GluC digestion demonstrated covalent engagement of 25 with a solvent exposed cysteine residue (Cys341). This secondary covalent engagement is likely responsible for the “double engagement” peak in FIG. 14 (SEQ ID NO: 2).

FIG. 17 illustrates the raw ScanEDGE data.

FIG. 18 illustrates the unprocessed immunoblot data of, in order, p-ATF2, total ATF2, beta-actin loading control, pJNK (sorbitol control lane included), total JNK (sorbitol control lane included), pMAP2K7 (sorbitol control lane included), and total MAP2K7 (sorbitol control lane included).

FIG. 19 illustrates the DK2403 and erlotinib cytotoxic effects. Erlotinib inhibits cell viability in the KOPT-K1 cell line at higher doses than DK2403, suggesting that inhibition of DK2403-mediated cytotoxicity is caused mainly by MAP2K7-JNK inhibition.

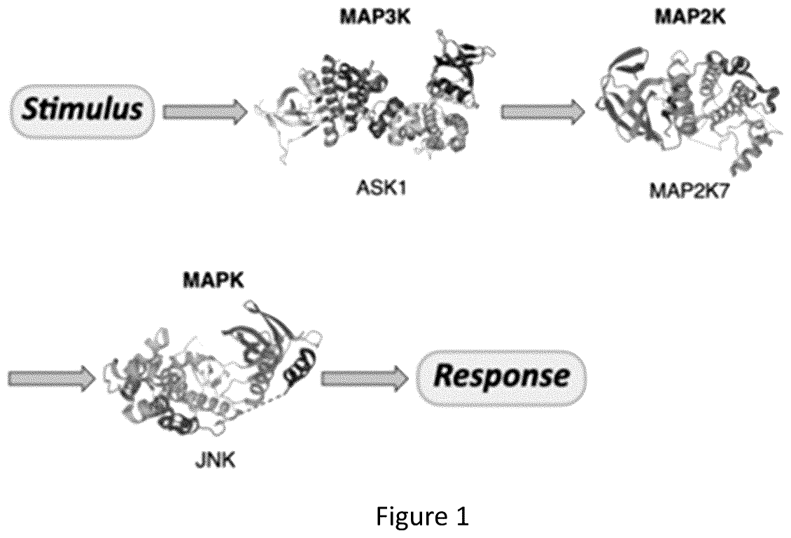

### DETAILED DESCRIPTION OF THE INVENTION

Disclosed herein are phenoxypyrimidine compounds and derivative thereof for use as inhibitors of mitogen-activated protein kinase 7 (MEK7). Diseases and disorders treated by the disclosed compounds, pharmaceutical compositions, and methods may include, but are not limited to, cell proliferative diseases and disorders such as cancer, including acute lymphoblastic leukemia (ALL), and specifically pediatric T-cell acute lymphoblastic leukemia.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Chemical Entities

The term “alkyl” refers to a straight-chain or branched alkyl radical in all of its isomeric forms, such as a straight or branched group of 1-12, 1-10, or 1-6 carbon atoms, referred to herein as C₁-C₁₂-alkyl, C₁-C₁₀-alkyl, and C₁-C₆-alkyl, respectively.

The term “alkylene” refers to a diradical of straight-chain or branched alkyl group (e.g., a diradical of straight-chain or branched C₁-C₁₂ alkyl group). Exemplary alkylene groups include, but are not limited to —CH₂—, —CH₂CH₂—, —CH₂CH₂CH₂—, —CH(CH₃)CH₂—, —CH₂CH(CH₃)CH₂—, —CH(CH₂CH₃)CH₂—, and the like.

The term “alkenyl” refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The terms “alkoxy” or “alkoxyl” refers to an alkyl group, as defined above, having an oxygen radical attached thereto. Representative alkoxy groups include methoxy, ethoxy, tert-butoxy and the like.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, the cycloalkyl group is not substituted, i.e., it is unsubstituted.

The term “heterocycloalkyl” (or “heterocyclyl”) refers to a monovalent saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S.

The term “halo” refers to a halogen atom or halogen radical (e.g., —F, —Cl, —Br, or —I).

The term “haloalkyl” refers to an alkyl group that is substituted with at least one halogen. For example, —CH₂F, —CHF₂, —CF₃, —CH₂CF₃, —CF₂CF₃, and the like.

The term “aryl” refers to a carbocyclic aromatic group. The term “aryl” includes monocyclic ring systems, and polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aryl ring is unsubstituted. In certain embodiments, the aryl group is a 6-10 membered ring structure. Representative aryl groups include phenyl, naphthyl, anthracenyl, 1,3-benzodioxolyl and the like.

The substituents on the aryl (e.g. phenyl) rings in the compounds of the disclosure may be at ortho-, meta-, or para-positions.

The term “heteroaryl” refers to an aromatic 5- to 10-membered ring structure, alternatively 6- to 10-membered rings, whose ring structures include one to four heteroatoms, such as nitrogen, oxygen, and sulfur. The number of ring atoms in the heteroaryl group can be specified using Cx-Cx nomenclature where x is an integer specifying the number of ring atoms. For example, a C₃-C₇ heteroaryl group refers to an aromatic 3- to 7-membered ring structure containing one to four heteroatoms, such as nitrogen, oxygen, and sulfur. The term “heteroaryl” includes monocyclic ring systems, and polycyclic ring systems having two or more heterocyclic rings in which two or more carbon or heteroatom are common to two adjoining rings (the rings are “fused rings”) wherein at least one of the rings is a heterocyclic aromatic group and, e.g., the other ring(s) may be cycloalkyls, cycloalkenyls, cycloalkynyls, and/or aryls. Representative heteroaryl groups include pyridinyl, quinolinyl, furanyl, thionyl, indolyl, and the like.

The term “indolyl” refers to the radical

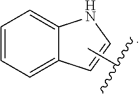

The term “1,3-benzodioxolyl” as used herein refers to the radical

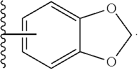

The term “vinyl” as used herein refers to the radical

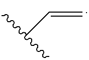 [SMILES: *C=C]

The term “propenyl” as used herein refers to the radical

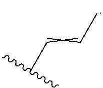 [SMILES: *C=CC]

The term “fluoromethyl” refers to the radical

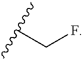 [SMILES: *CF]

The term “chloromethyl” refers to the radical

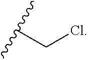 [SMILES: *CCl]

The term “optionally substituted” refers to one or more carbon atoms in the group being independently substituted with one or more functional groups described herein.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The present invention encompasses various stereo isomers of these compounds and mixtures thereof. Stereoisomers include enantiomers and diastereomers. Mixtures of enantiomers or diastereomers may be designated (±)″ in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essentially of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

As used herein, “salt” refers to acid addition salts and basic addition salts. It may also refer to those salts that may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### Compounds

Disclosed herein includes a compound of formula I, or a salt thereof:

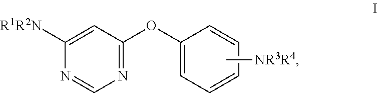

wherein
R¹ is hydrogen or alkyl;
   R² is selected from the group consisting of alkyl, cycloalkyl, heterocycloalkyl, -alkylene-heteroaryl, -alkylene-aryl, wherein aryl is optionally substituted with one or more substituents selected from the group consisting of halo, alkyl, haloalkyl, and alkoxy;
or —NR¹R² together form a heterocycloalkyl;
R³ is hydrogen or alkyl;
R⁴ is —C(O)R⁵; and
   R⁵ is alkenyl or alkyl optionally substituted with one or more substituents selected from halo and alkoxy.

In some embodiments, the compound of formula I has a formula I(a):

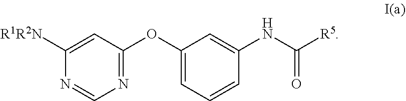

In some embodiments, R¹ is hydrogen, R² is alkyl or —(CH₂)ₙ-aryl, wherein aryl is optionally substituted with one or more halo, R⁵ is chloromethyl, and n is an integer of 1-6 in the compound of formula I(a).

In some such embodiments, R¹ is hydrogen, R² is methyl or —(CH₂)ₙ-phenyl, wherein phenyl is optionally substituted with one to three fluoro, R⁵ is chloromethyl, and n is an integer of 1-3 in the compound of formula I(a).

In some such embodiments, the compound of formula I(a) is

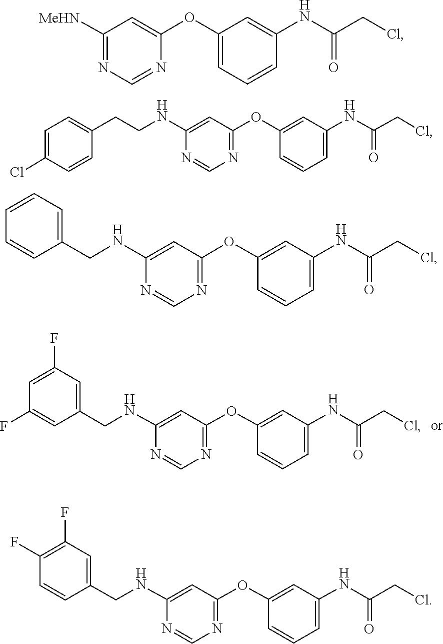

In some embodiments, R¹ is hydrogen or methyl, R² is selected from alkyl, C₃-C₈-cycloalkyl, —(CH₂)ₙ-aryl, and —(CH₂)ₙ-heteroaryl, wherein aryl is optionally substituted with one or more substituents selected from the group consisting of alkyl, alkoxy, halo, and haloalkyl, or —NR¹R² together form a heterocycloalkyl containing 1-3 heteroatoms selected from N, O, and S, R⁵ is vinyl or chloromethyl, and n is an integer of 1-6 in the compound of formula I(a).

In some such embodiments, R¹ is hydrogen or methyl, R² is selected from methyl, C₃-C₆-cycloalkyl, —(CH₂)ₙ-phenyl, and —(CH₂)ₙ—(C₇-C₁₀-heteroaryl), wherein phenyl is optionally substituted with one to three substituents selected from methyl, methoxy, fluoro, and trifluoromethyl, or —NR¹R² together form a heterocycloalkyl containing 1-2 heteroatoms selected from N and O, R⁵ is vinyl or chloromethyl, and n is an integer of 1-3 in the compound of formula I(a).

In some such embodiments, the compound of formula I(a) is

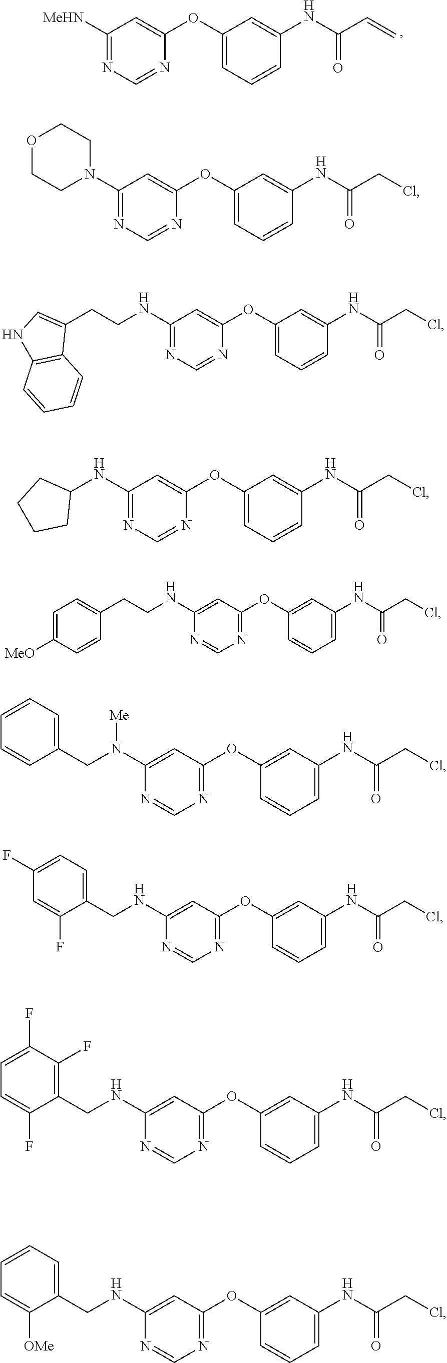

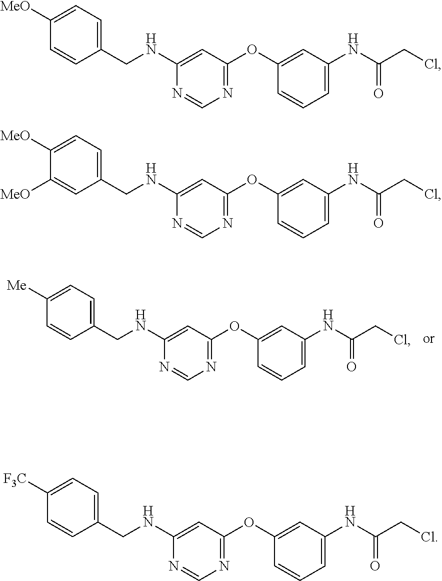 [SMILES: COc1ccc(CNc2cc(Oc3cccc(NC(=O)CCl)c3)ncn2)cc1 ; COc1ccc(CNc2cc(Oc3cccc(NC(=O)CCl)c3)ncn2)cc1OC ; Cc1ccc(CNc2cc(Oc3cccc(NC(=O)CCl)c3)ncn2)cc1 ; "or" ; O=C(CCl)Nc1cccc(Oc2cc(NCc3ccc(C(F)(F)F)cc3)ncn2)c1]

In some embodiments, R¹ is hydrogen, R² is selected from alkyl, heterocycloalkyl containing 1-3 heteroatoms selected from N, O, and S, and —(CH₂)ₙ-aryl, wherein aryl is optionally substituted with one or more substituents selected from the group consisting of halo, alkoxy, and haloalkyl, R⁵ is fluoromethyl or chloromethyl and n is integer of 1-6 in the compound of formula I(a).

In some such embodiments, R¹ is hydrogen, R² is selected from methyl, heterocycloalkyl containing 1-2 heteroatoms selected from N and O, —(CH₂)ₙ-benzodioxolyl, and —(CH₂)ₙ-phenyl, wherein phenyl is optionally substituted with one to three substituents selected from fluoro, methoxy, and trifluoromethyl, R⁵ is fluoromethyl or chloromethyl and n is integer of 1-3 in the compound of formula I(a).

In some such embodiments, the compound of formula I(a) is

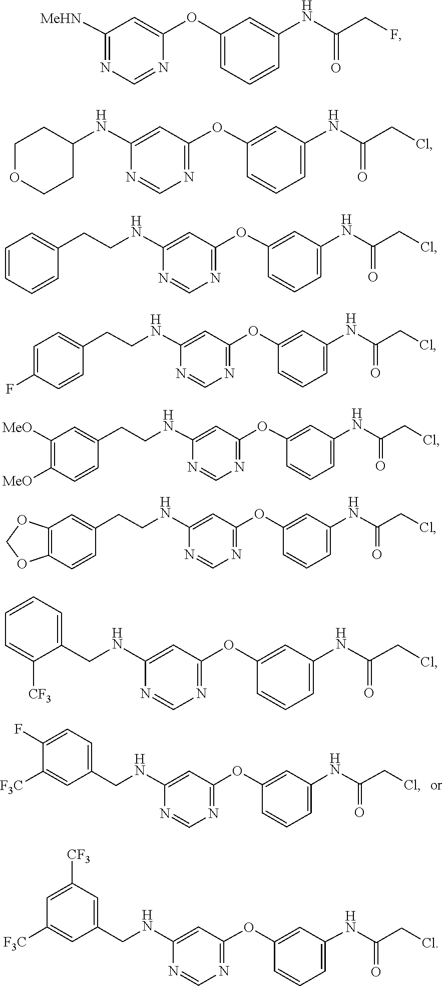

In some embodiments, R¹ is hydrogen, R² is alkyl, and R⁵ is propenyl or alkyl substituted with chloro or methoxy in the compound of formula I(a). In some such embodiments, the compound of formula I(a) is

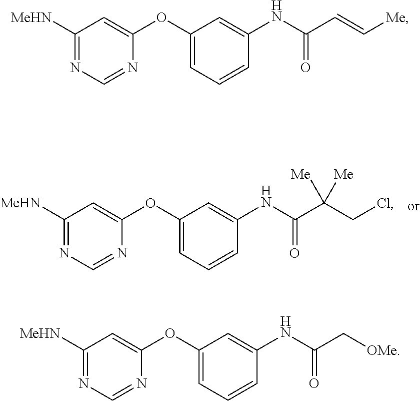 [SMILES: C/C=C/C(=O)Nc1cccc(Oc2cc(NC)ncn2)c1 ; CNc1cc(Oc2cccc(NC(=O)C(C)(C)CCl)c2)ncn1 ; CNc1cc(Oc2cccc(NC(=O)COC)c2)ncn1]

In some embodiments, the compound of formula I has a formula I(b):

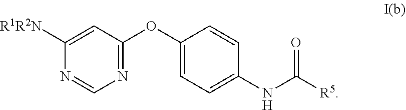

In some embodiments, R¹ is hydrogen, R² is alkyl, and R⁵ is vinyl or chloromethyl in the compound of formula I(b). In some such embodiments, the compound of formula I(b) is

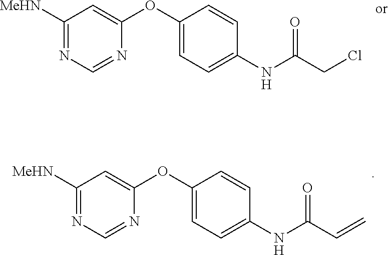 [SMILES: CNc1cc(Oc2ccc(NC(=O)CCl)cc2)ncn1 ; C=CC(=O)Nc1ccc(Oc2cc(NC)ncn2)cc1]

The disclosed compounds may exhibit one or more biological activities. In some embodiments, the disclosed compounds modulate the activity of mitogen-activated protein kinase 7 (MEK7). In some embodiments, the disclosed compounds inhibit the activity of MEK7 by at least 50%, 60%, 70%, 80%, 90%, 95%, 96%, 97%, 98%, or 99% at a concentration of less than 100 M, 50 M, 10 μM, 1 μM, 0.1 μM, 0.05 UM, 0.01 μM, 0.005 UM, 0.001 μM, or less. The disclosed compounds may inhibit the growth of cells that express MEK7 (preferably by at least 50%, 60%, 70%, 80%, 90%, 95%, 96%, 97%, 98%, or 99% at a concentration of less than about 100 μM, 50 M, 10 μM, 1 μM, 0.1 μM, 0.05 μM, 0.01 μM, 0.005 UM, 0.001 UM, or less). The disclosed compounds may not inhibit the growth of cells that do not express MEK7 (preferably by not more than 50%, 40%, 30%, 20%, 10%, 5%, 4%, 3%, 2% or less at a concentration of greater than about 0.001 μM, 0.005 UM, 0.01 μM, 0.5 μM, 0.1 μM, 1.0 μM, 10 μM, and 100 μM or higher). Concentration ranges also are contemplated herein, for example, a concentration range bounded by end-point concentrations selected from 0.001 μM, 0.005 UM, 0.01 μM, 0.5 μM, 0.1 μM, 1.0 μM, 10 UM, and 100 μM.

In some embodiments, the disclosed compounds are selective MEK7 inhibitors. The term “selective MEK7 inhibitor” refers to a compound that has an IC₅₀ in inhibiting MEK7 that is at least 10 times greater, 50 times greater, 100 times greater, 200 times greater, 300 times greater, 500 times greater, 800 times greater, 1000 times greater, 2000 times greater, 3000 times greater, 4000 times greater, or 5000 times greater, than its IC₅₀ in inhibiting other MEK isoforms, including MEK1, MEK2, MEK3, MEK4, MEK5, and/or MEK6.

Cell proliferation and inhibition thereof by the disclosed compounds may be assessed by cell viability methods disclosed in the art including ADP-Glo kinase assay to assess cell viability. In some embodiments, the disclosed compounds have an IC₅₀ from greater than 0 μM to 50 μM, from greater than 0 μM to 40 μM, from greater than 0 μM to 30 μM, from greater than 0 M to 20 μM, from greater than 0 μM to 10 μM, from greater than 0 μM to 5 μM, from greater than 0 μM to 1 μM, from greater than 0 μM to 0.5 μM, from greater than 0 μM to 0.1 μM, or from greater than 0 μM to less than 0.1 μM, in ADP-Glo kinase assay.

### Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

In some embodiments, the compounds disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 UM, and 100 μM (e.g., 0.1 μM-1.0 μM).

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

In some embodiments, a pharmaceutical composition comprising the compound of as disclosed herein and a pharmaceutically suitable carrier, diluent, or excipient is provided.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 UM, 0.005 UM, 0.01 μM, 0.5 μM, 0.1 μM, 1.0 μM, 10 μM, and 100 μM (e.g., 0.1 μM-1.0 μM).

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Acrosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Optionally, the disclosed compounds or pharmaceutical compositions comprising the disclosed compounds may be administered with additional therapeutic agents, optionally in combination, in order to treat cell proliferative diseases and disorders. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed compounds and further to comprise one or more additional therapeutic agents, for example, one or more additional therapeutic agents for treating cell proliferative diseases and disorders.

Methods of preparing pharmaceutical formulations or compositions include the step of bringing an inhibitor compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

Regardless of composition or formulation, those skilled in the art will recognize various avenues for medicament administration, together with corresponding factors and parameters to be considered in rendering such a medicament suitable for administration.

### Methods

The disclosed compounds and pharmaceutical compositions comprising the disclosed compounds may be administered in methods of treating a subject in need thereof. For example, in the methods of treatment a subject in need thereof may include a subject having a cell proliferative disease, disorder, or condition such as cancer.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

A “subject in need thereof” as utilized herein refers to a subject in need of treatment for a disease or disorder associated with mitogen-activated protein kinase 7 (MEK7) activity. The term “subject” may be used interchangeably with the terms “individual” and “patient” and includes human and non-human mammalian subjects. In some embodiments, the treated subject may be a mammalian subject. Although the methods disclosed herein are particularly intended for the treatment of proliferative disorders in humans, other mammals are included. By way of non-limiting examples, mammalian subjects include monkeys, equines, cattle, canines, felines, mice, rats and pigs.

As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

Diseases and disorders associated with MEK7 activity may include, but are not limited to, cell proliferative diseases and disorders such as cancer. Cancers may include, but are not limited to, acute lymphoblastic leukemia, pediatric T-cell acute lymphoblastic leukemia, adrenal cancer, anal cancer, bladder cancer, bone cancer, breast cancer, cervical cancer, Chronic Lymphocytic Leukemia (CLL), Chronic Myeloid Leukemia (CML), Chronic Myelomonocytic Leukemia (CMML), colorectal cancer, endometrial cancer, esophagus cancer, gallbladder cancer, carcinoid tumors, kidney cancer, leukemia, liver cancer, lung cancer, lymphoma, nasopharyngel cancer, non-small cell lung cancer, ovarian cancer, pancreatic cancer, penile cancer, pituitary tumors, prostate cancer, skin cancer (especially melanoma), small cell lung cancer, small intestine cancer, stomach cancer, testicular cancer, thymus cancer, thyroid cancer, vaginal cancer, and vulvar cancer. In some embodiments, the cancer is pediatric T-cell acute lymphoblastic leukemia.

The compounds for use according to the methods of disclosed herein may be administered as a single compound or a combination of compounds. For example, a compound that modulates MEK7 activity may be administered as a single compound or in combination with another compound that modulates MEK7 activity or that has a different pharmacological activity.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg).

In some embodiments of the disclosed treatment methods, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with MEK7 activity.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

The compounds and compositions disclosed herein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

### Miscellaneous

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

Krüppel-like factor 4 (KLF4) is a zinc-finger transcription factor¹² known to function as either an oncogene or a tumor suppressor in a context-dependent manner.¹³⁻¹⁴ Epigenetic silencing of Klf4 by CpG methylation occurs in pediatric acute lymphoblastic leukemia (ALL), and decreased KLF4 expression has been identified in treatment-resistant cases of ALL.¹⁵⁻¹⁶ Physiologically, KLF4 represses transcription of Map2k7, a gene encoding mitogen-activated protein kinase kinase 7 (MAP2K7), also known as MAPK/Erk kinase 7 (MEK7, or MKK7).¹¹ Loss of Map2k7 repression and consequent amplification of downstream MAPK signaling may contribute to T-cell ALL (T-ALL) pathology.^{11,17} Inhibition of MEK7¹⁸⁻¹⁹ or its substrate c-Jun N-terminal kinase (JNK) 11 may reduce leukemic expansion in patient-derived xenograft mouse models.

JNK is a substrate of MAP2K7,^{11,20-22} but it is also implicated in genomic stability through its roles in the repair of DNA double-strand breaks²³ and photodamage,²⁴ which are not known to be MAP2K7-dependent. Direct inhibition of MAP2K7 would facilitate the investigation of Klf4 inactivation and consequent MAPK signaling amplification in T-ALL.²² Further, despite sharing structural homology and a common phosphorylation substrate in JNK, MAP2K4 and MAP2K7 exhibited strikingly dissimilar susceptibility to inhibitors previously studied^{33,34}.

Structural analysis of MEK ATP binding pockets identified MAP2K7 to have the shallowest depth and smallest volume among all isoforms with available crystal structures.³³ MAP2K7 is also unique among the MEK family for the presence of four cysteine residues in its active site.³³ It is the only MEK isoform featuring a hinge region cysteine (Cys218),³³ and only 11 human kinases bear a cysteine at the same relative position.³⁸

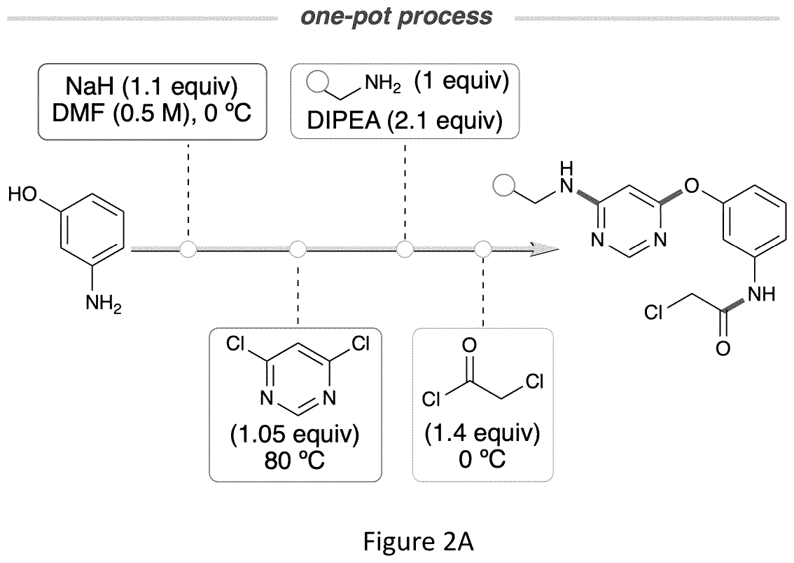

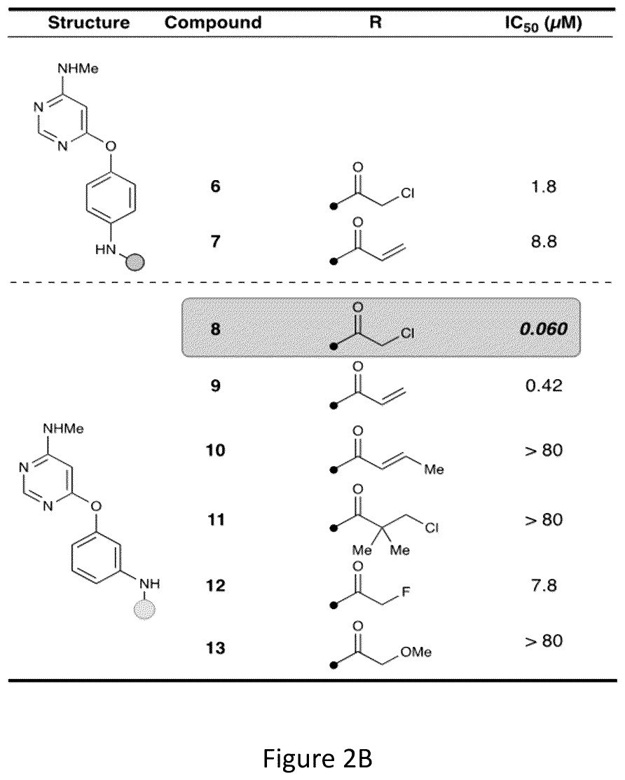

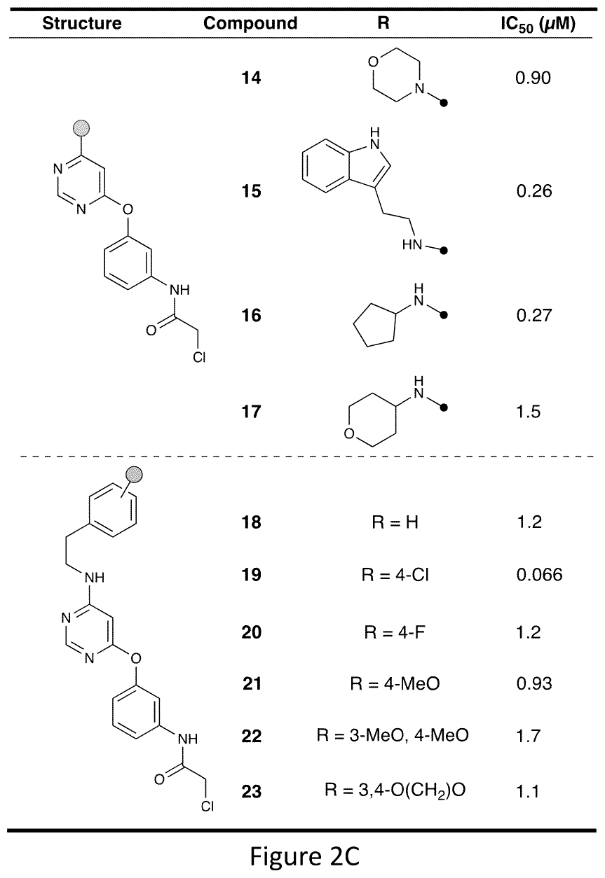

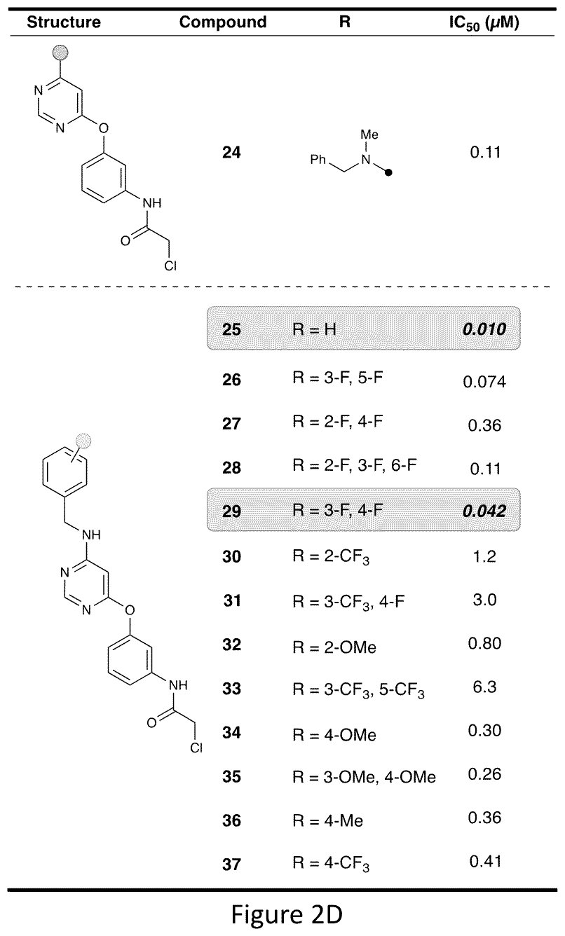

A streamlined synthesis was developed to access MEK7 inhibitors. Each compound was prepared by a one-pot process which afforded the desired MAP2K7 inhibitor following workup and chromatography (FIG. 2A). This expedient and high-yielding procedure enabled the rapid generation of chemical diversity.

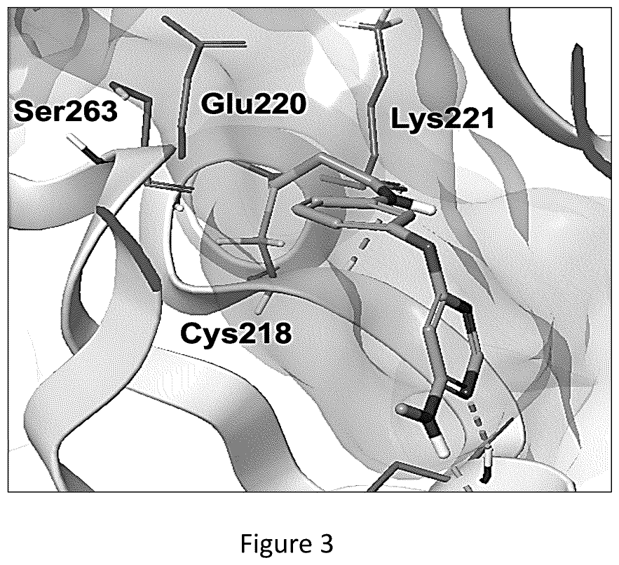

With preliminary derivatives and controls in hand, we commenced their biological characterization by surveying in vitro activity against MAP2K7. As shown in FIG. 2B, 8 demonstrated inhibitory capacity, having an IC₅₀ of 60 nM. Compounds 6, 7, and 9 were less active than compound 8. To probe steric congestion in the proximity of the electrophile, compounds 10 and 11 were prepared and assayed. Computational modeling of 7 suggested a great deal of steric congestion in proximity to Cys218 (FIG. 3). Exchanging the electrophilic α-chloroacetamide for the α-fluoroacetamide (12) or α-methoxyacetamide (13) resulted in attenuated activity, implying a covalent mechanism.

Greater potency was endowed by the α-chloroacetamide electrophilic motif and meta-substitution around the arene. Although all FDA-approved covalent inhibitors to date feature electrophilic acrylamides or epoxyketones,⁴¹ the half-life of α-chloroacetamides surpasses that of acrylamides in the presence of glutathione at physiological temperature and pH.⁴²

We next probed the chemical space with respect to aminopyrimidine functionality. The chloropyrimidine intermediate (SI-1) was derivatized with a variety of phenethylamines, other primary amines, as well as secondary amines (FIG. 2C). As a whole, compounds with less steric congestion in close proximity to the pyrimidine fared better, with select arylmethylamine-derived inhibitors (15 and 19) demonstrating the greatest potency. Even moderately sterically-demanding substitution along the appendant arene was associated with lowered potency (e.g., 21-23).

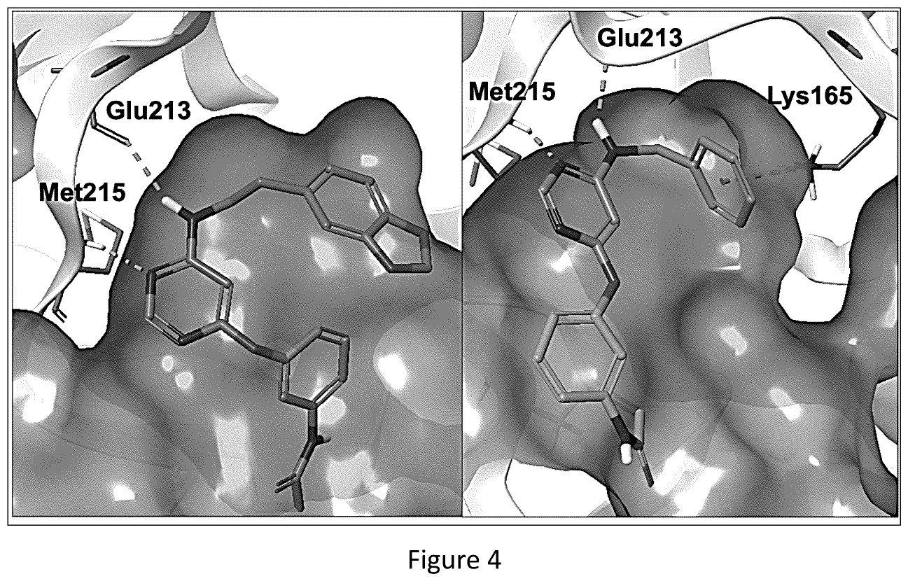

These findings were paralleled by docking studies (PDB: 6YG3):³² substituted phenethylamines suffered from steric bulk at the non-electrophilic end of the molecule without gaining additional favorable interactions. This observation is in stark contrast with benzylamine-derived inhibitors, which computational studies predicted to engage in an additional cation-π interaction with Lys165 (FIG. 4). This interaction additionally appears to steer the carbon skeleton of the inhibitor away from unfavorable steric interactions.

A class of benzylamine-functionalized pyrimidines were synthesized and evaluated (FIG. 3D). Compound 25 (DK2403) demonstrated extremely high potency (IC₅₀=10 nM, IC₅₀=93 without preincubation,⁴⁶ see Experimental section and Figures). The potency of its N-methylated derivative (24) suffered ten-fold attenuation. Arene substitution may lead to a decrease in potency, with some positions and substitutions tolerated better than others.

Small substituents such as fluorine atoms were well tolerated (26-29), with the 3,4-difluorinated derivative (29) demonstrating 42 nM potency. Substituents as large as a methyl group led to decreased potencies, and substitution at the ortho position (30, 32) pushed potencies towards the micromolar range. Larger substituents were better tolerated in the para (34, 36) and meta (35) positions, although di-substitution at the meta positions was poorly tolerated (33).

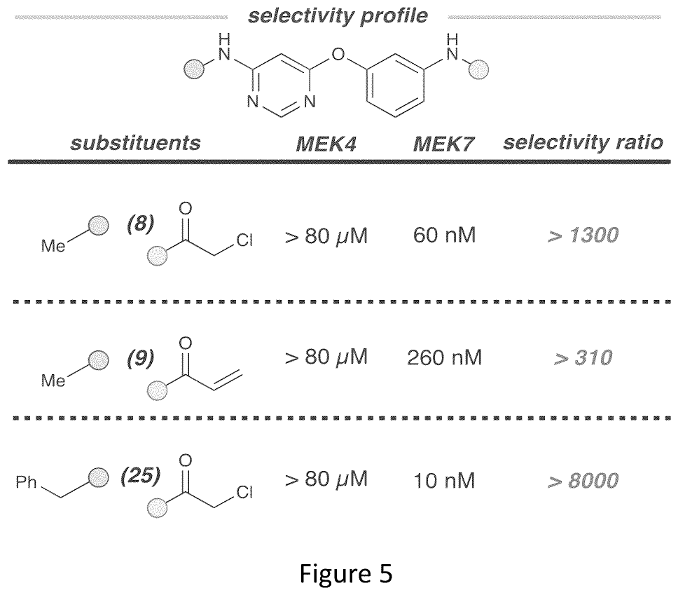

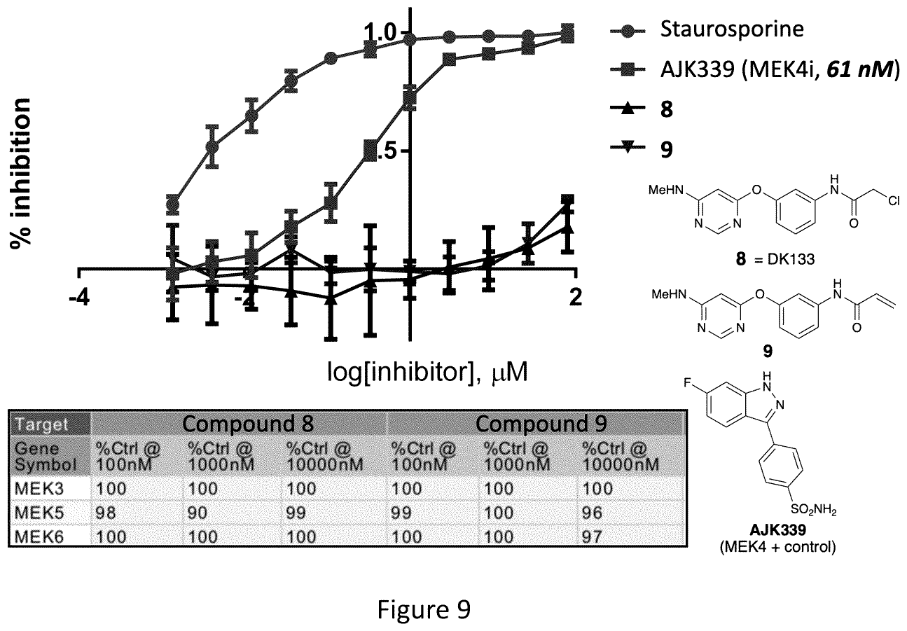

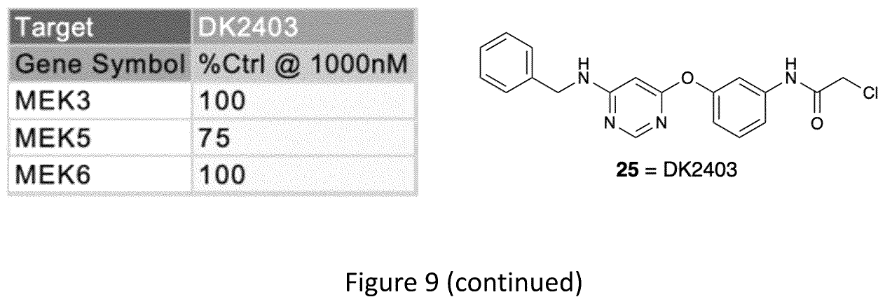

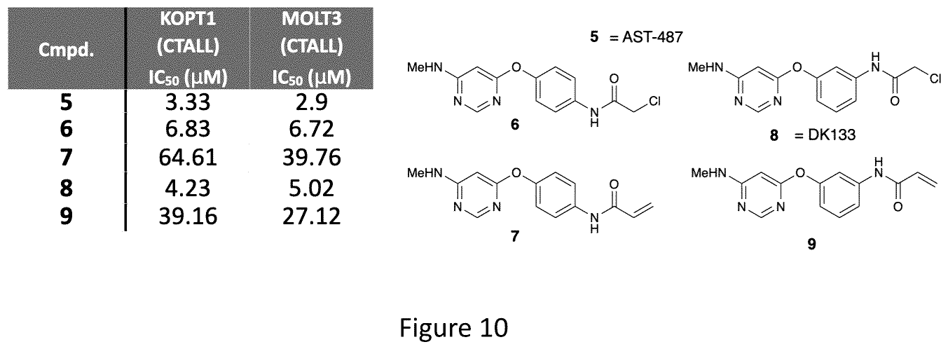

With a series of highly potent MEK7 inhibitors in hand, we next probed the selectivity of these compounds. Among the MEK isoforms, MEK7 shares the greatest structural homology with MEK4, making it the key kinase to test as we commence selectivity studies.⁴⁷⁻⁴⁸ Potency was determined against MEK4 employing our standard protocol. Compounds 8, 9, and 25 showed little activity against MEK4 (IC₅₀>80 μM, FIG. 5). When studied against the MEK3, 5, and 6 isoforms, 8, 9, and 25 were again found to be inactive (See FIG. 9). MEK4 and MEK7 feature high structural³³ and functional²¹ homology, and selectively inhibiting one in the presence of the other is often difficult.^{31,47} We were surprised to not induce MEK4 inhibition at concentrations as high as 80 μM, prompting us to pursue further selectivity studies.

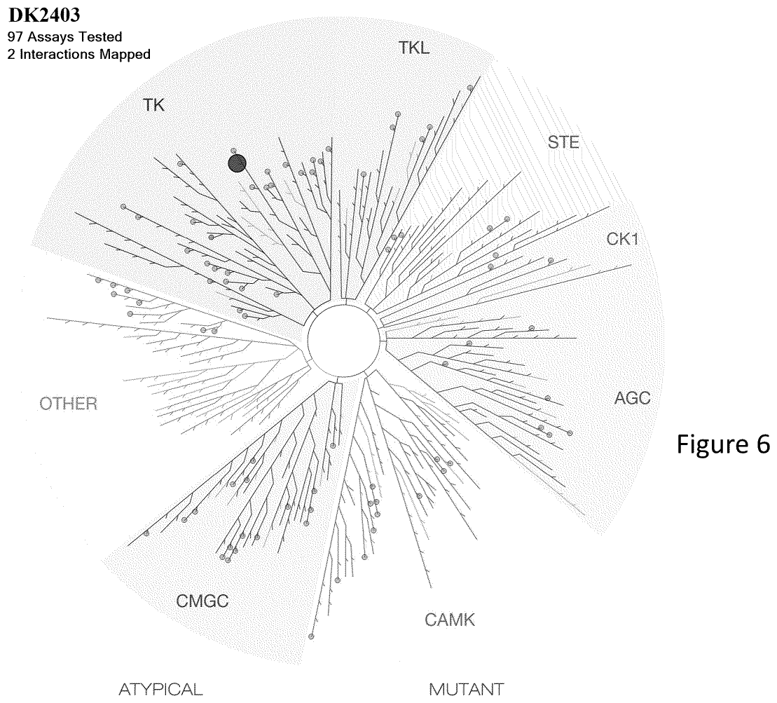

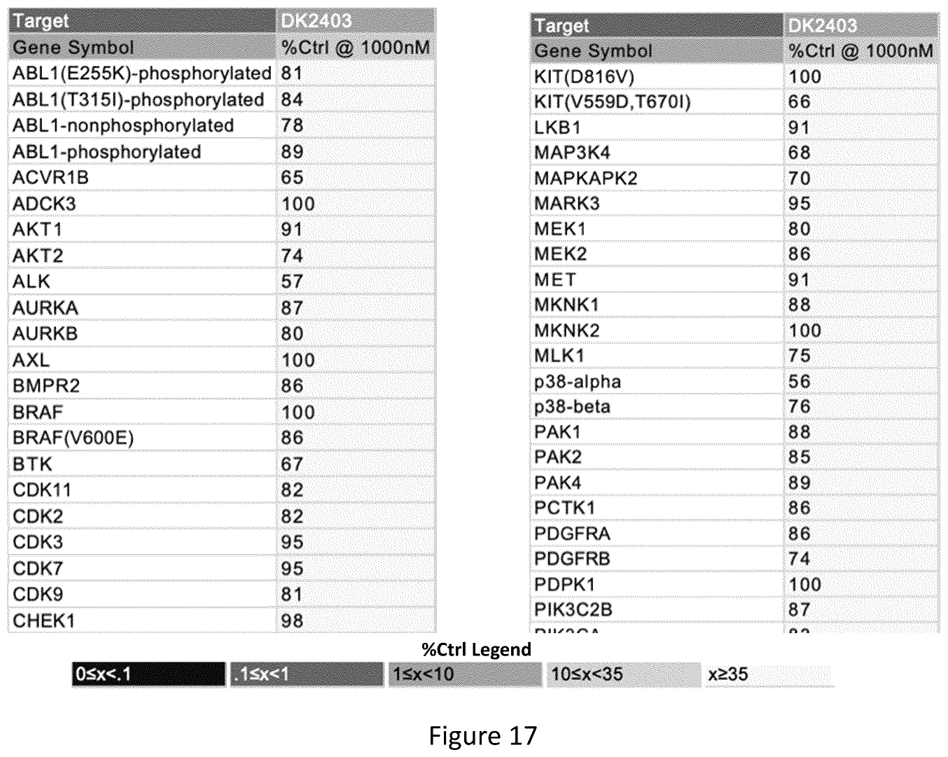

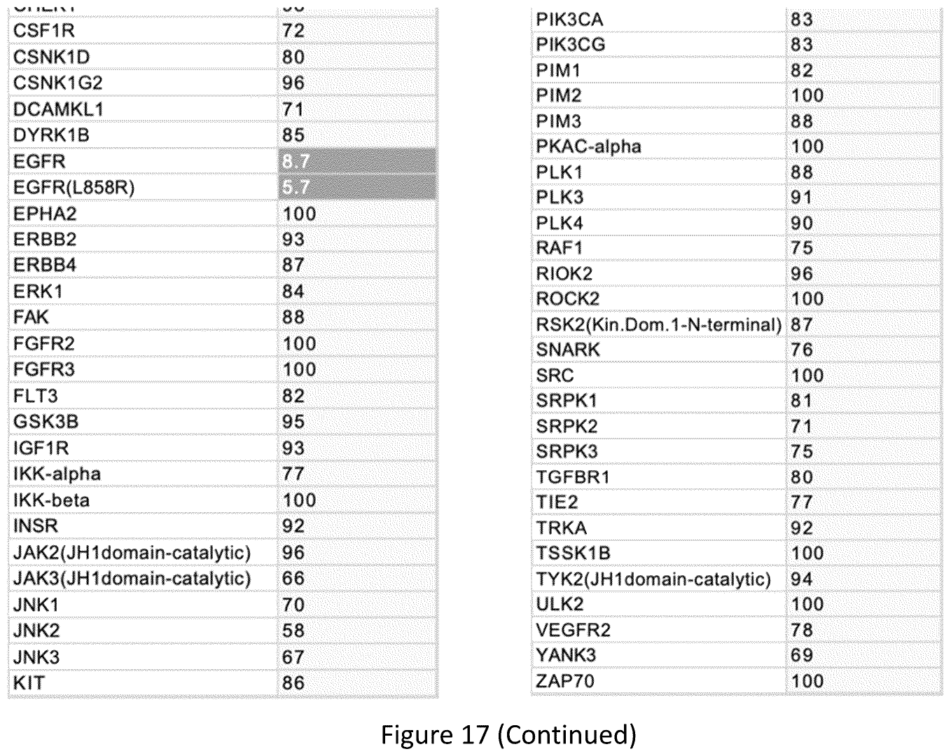

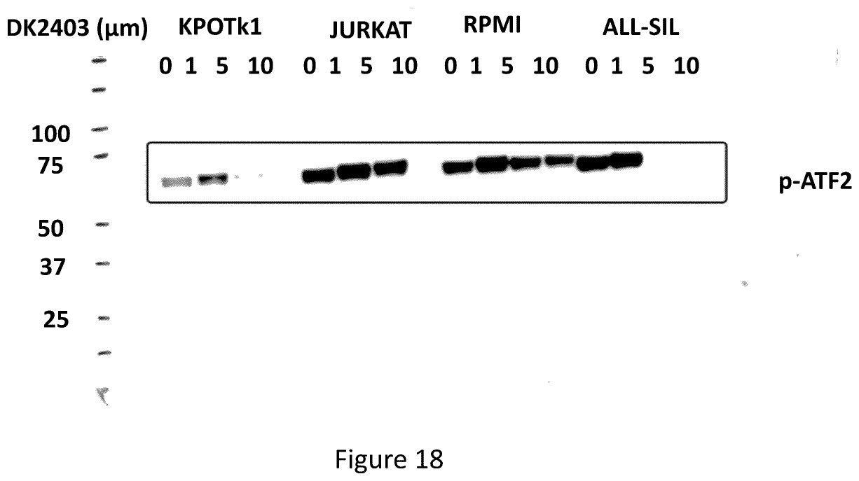

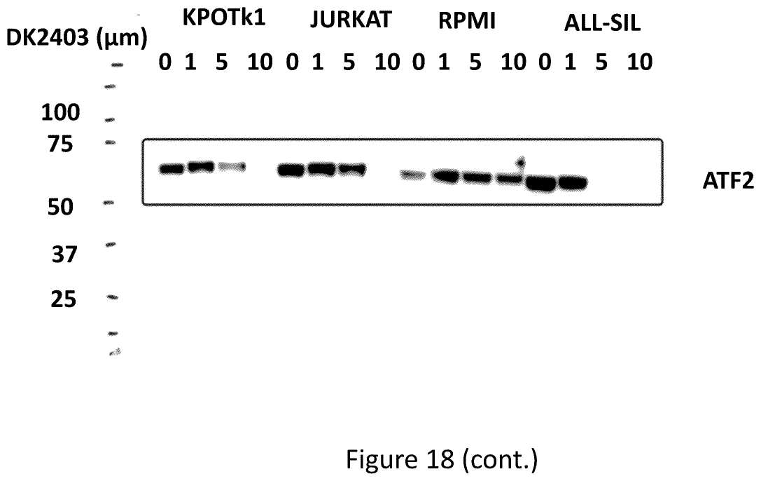

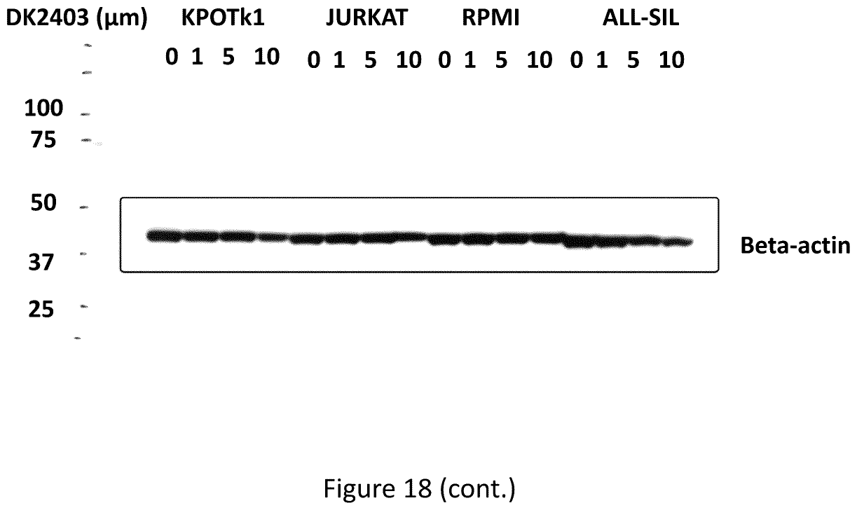

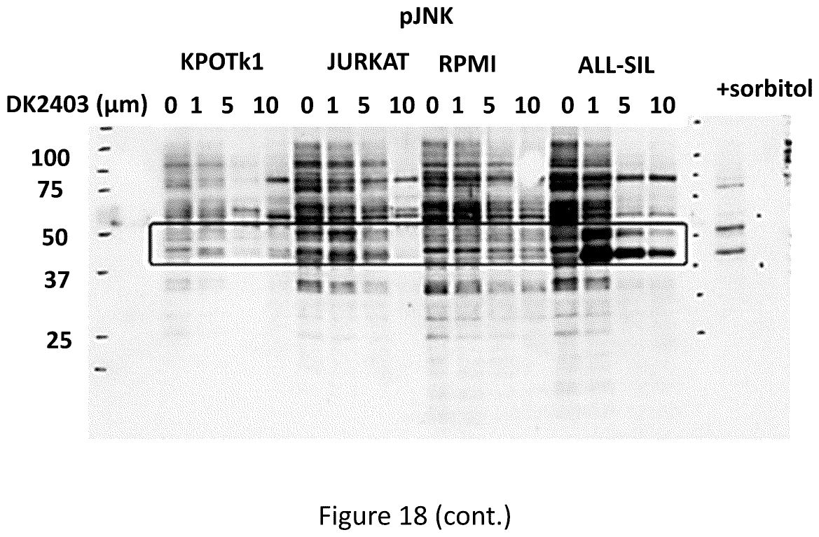

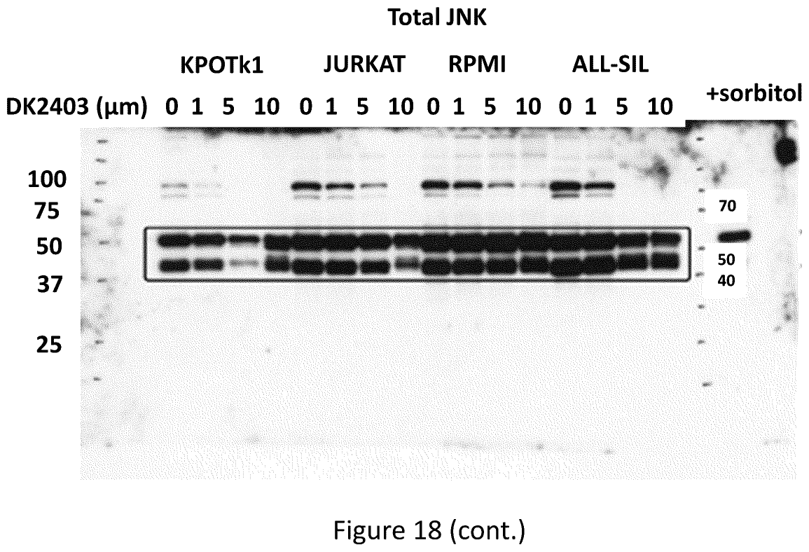

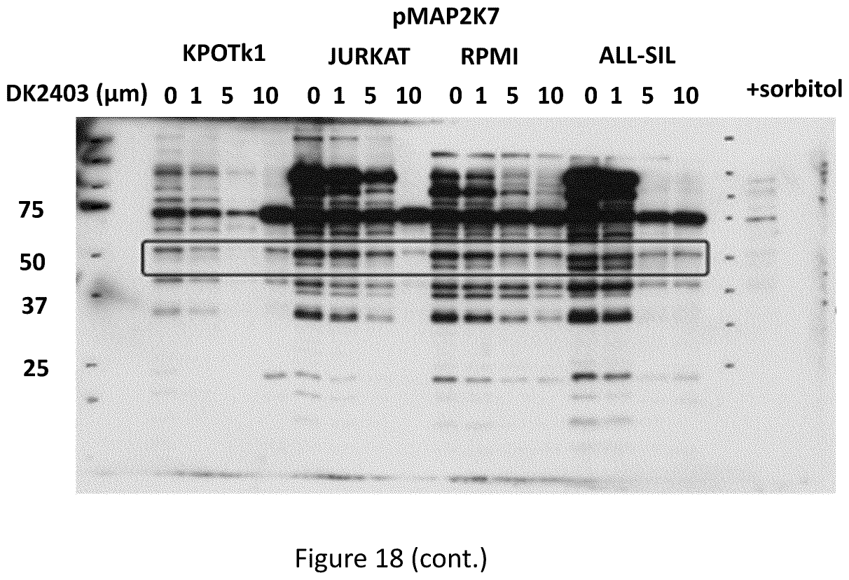

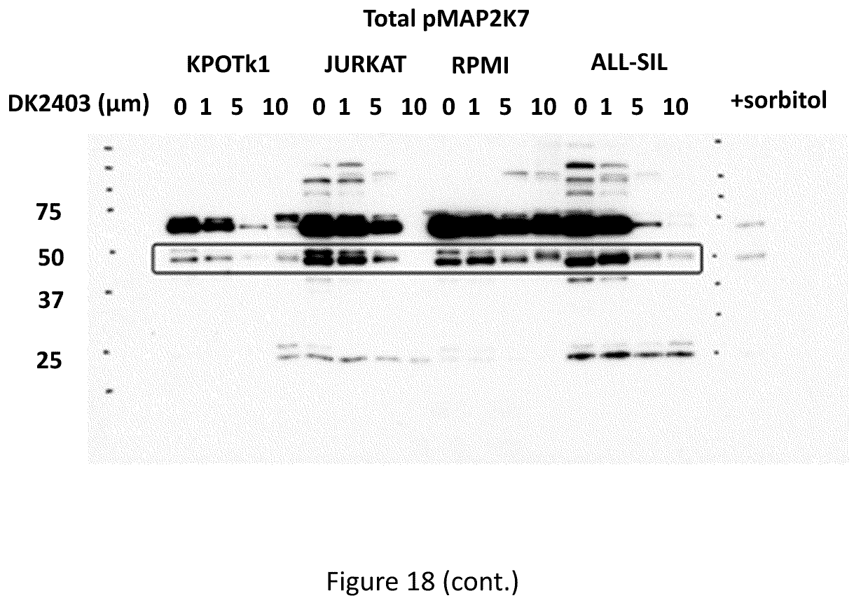

To better understand the selectivity profile of this class of inhibitors, 25 was subjected to a 97-kinase selectivity screen (FIG. 6, ScanEDGE, DiscoverX/Eurofins; see FIG. 17 for details). Among the 97 off-target kinases surveyed, our lead compound only interacted significantly with EGFR and the EGFR (L858R) mutant at 1 M (selectivity scores:⁴⁹ S (35)=S (10)=0.011). Additional assays confirmed lack of binding at MEK3, MEK5, and MEK6 (FIG. 9). Notably, 25 has no appreciable activity on MEK1/2, p38α/β, or JNK1/2/3, which are relevant kinases in our model for molecular pathophysiology in T-ALL.^{11,17-18,50} This compound also does not engage FLT3, a potential anticipated pitfall given the structural similarities between 25 and 5, a reversible FLT3 inhibitor. MAP2K7 is not among the 90 wildtype kinases assayed and represented in FIG. 6.

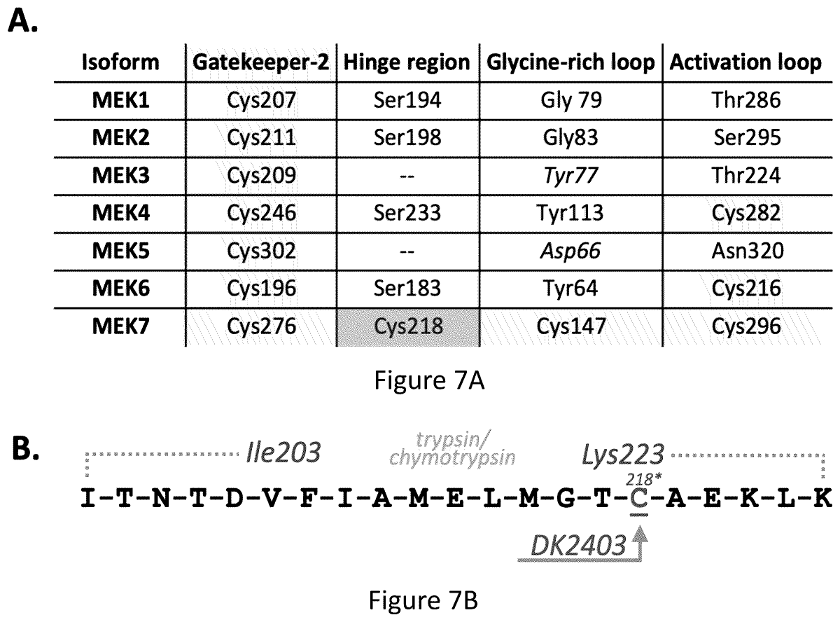

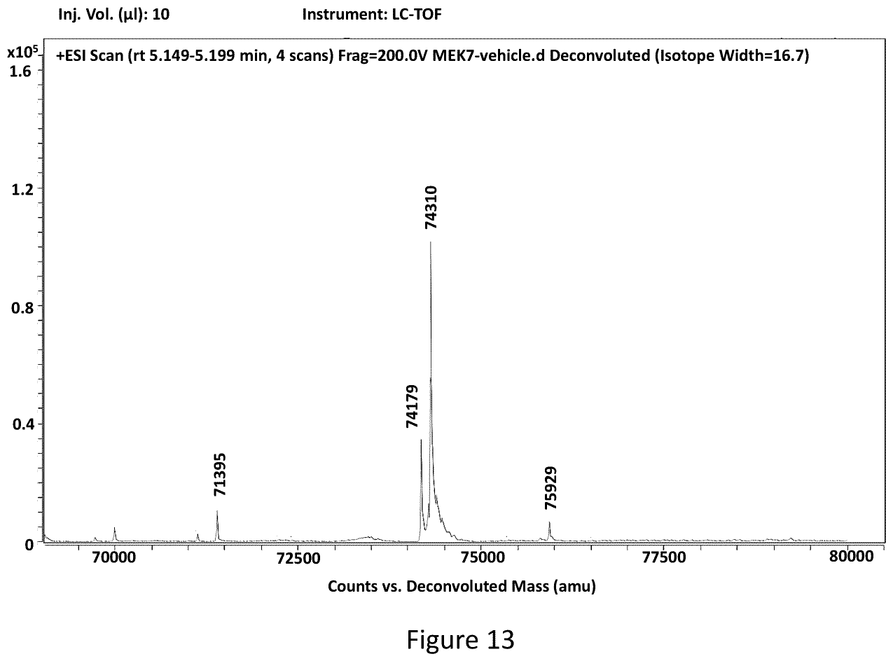

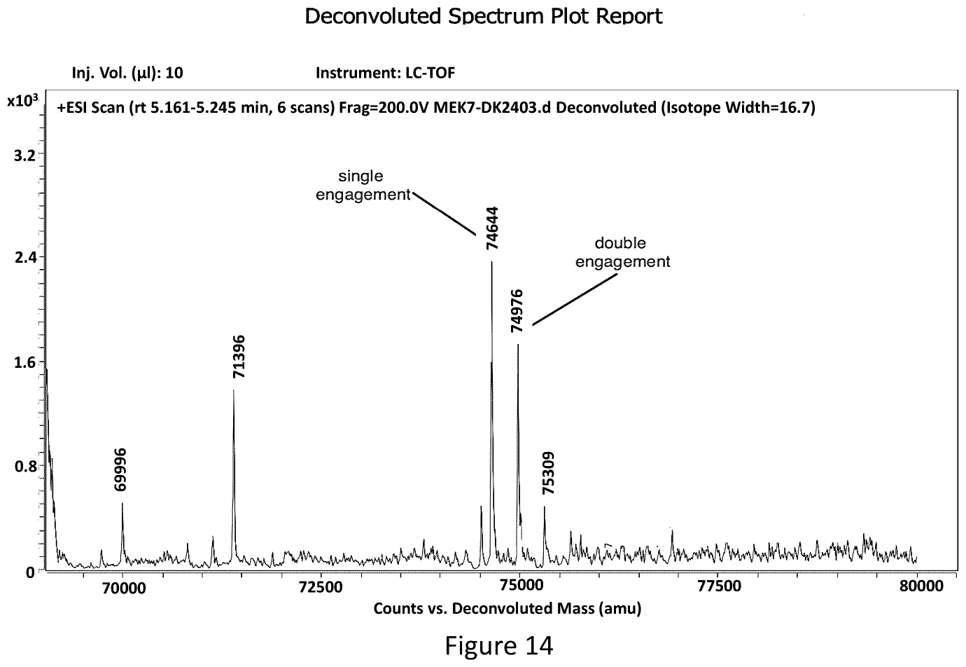

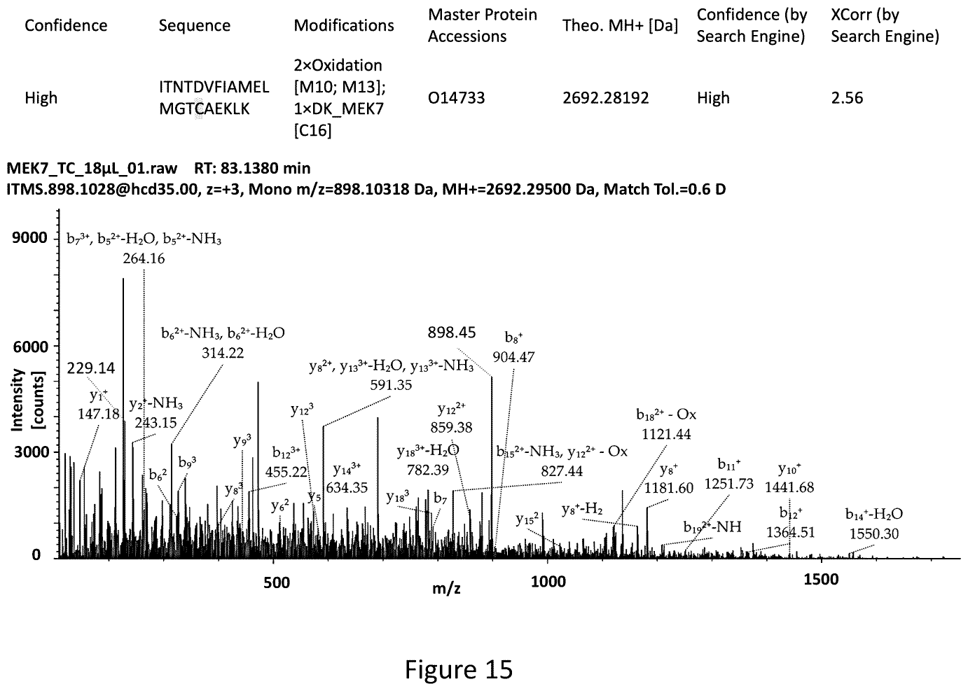

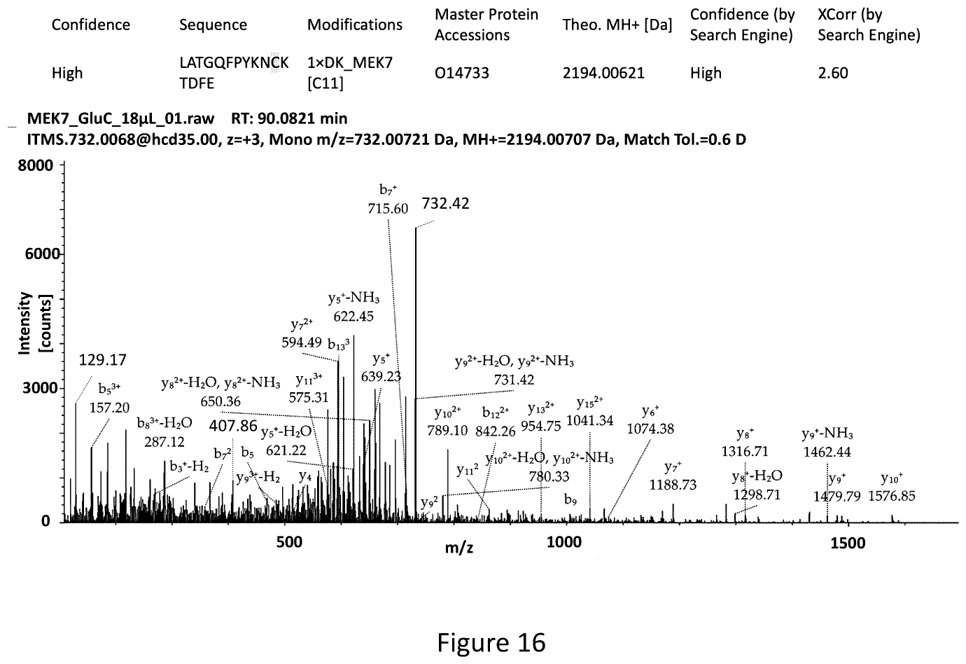

To confirm the suspected covalent mechanism of DK2403 (25), full-length MAP2K7 N-terminal GST fusion protein (74 kDa) was incubated with 25 (2 equiv), and the incubate was analyzed by LC-TOF MS. To our delight, MS analysis demonstrated covalent engagement (see FIGS. 13 and 14). The incubates were then digested with either trypsin/chymotrypsin combination or GluC. The former digest revealed covalent engagement of 25 with the intended Cys218 residue (FIGS. 7A-7B and 15). Adducts involving other active site residues were not observed, although labeling of solvent-exposed Cys341 was observed following GluC digestion (FIG. 16). Selectivity of our most potent compound likely derives from targeting Cys218, as only 11 human kinases bear an analogous cysteine (FIG. 7A).³⁸

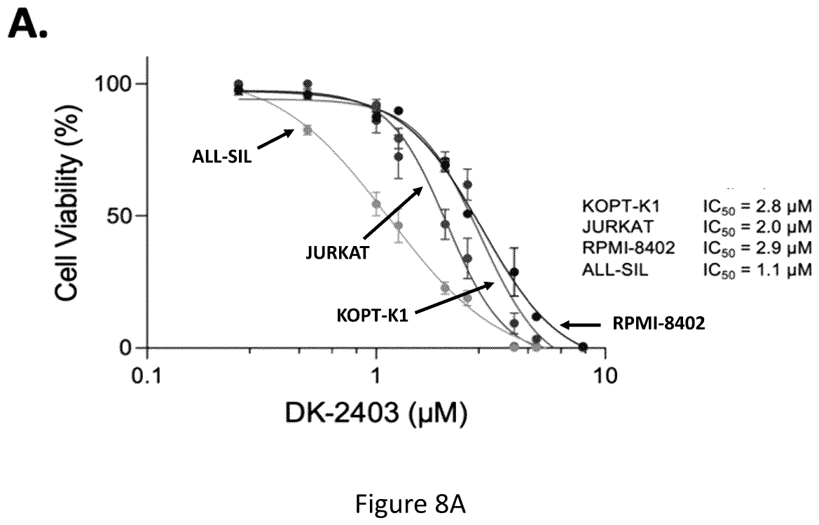

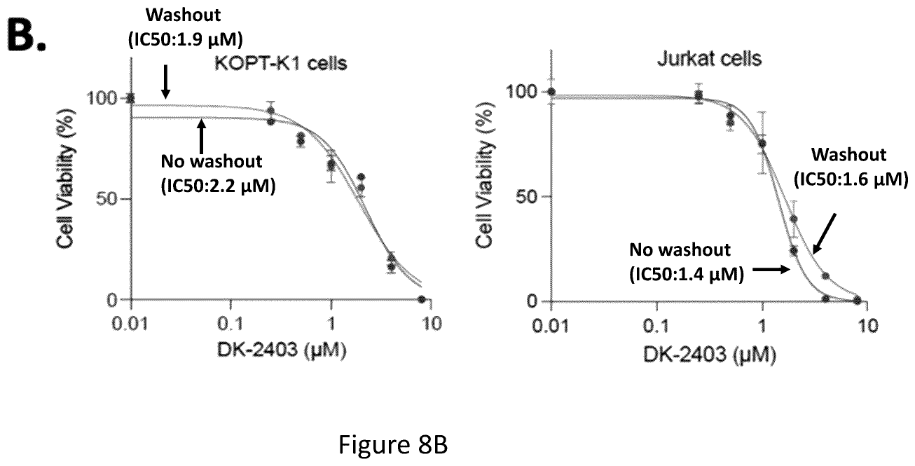

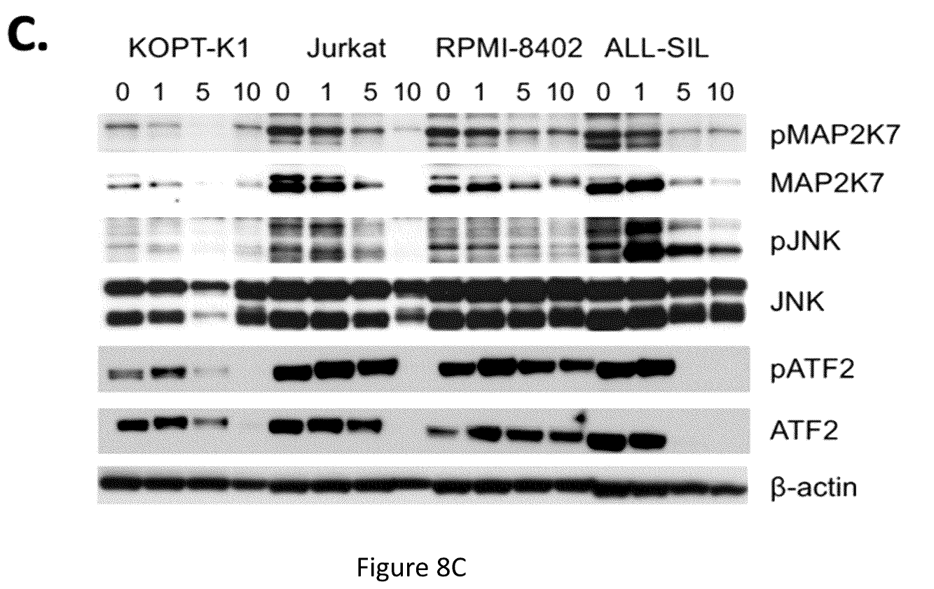

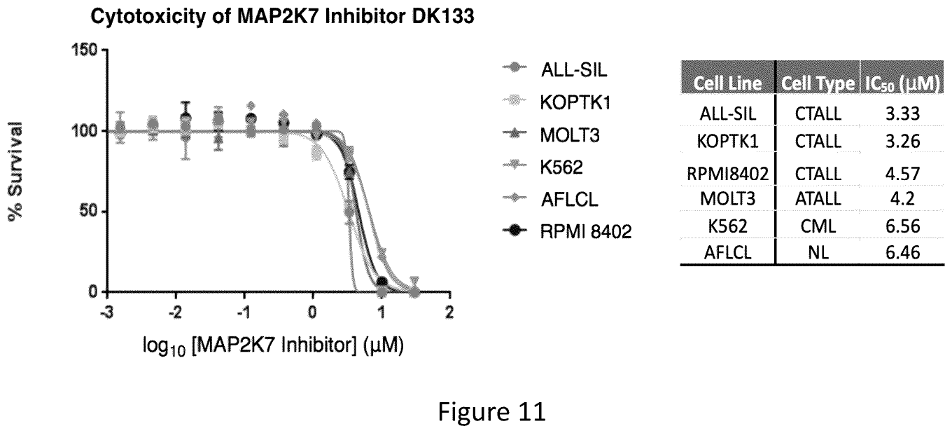

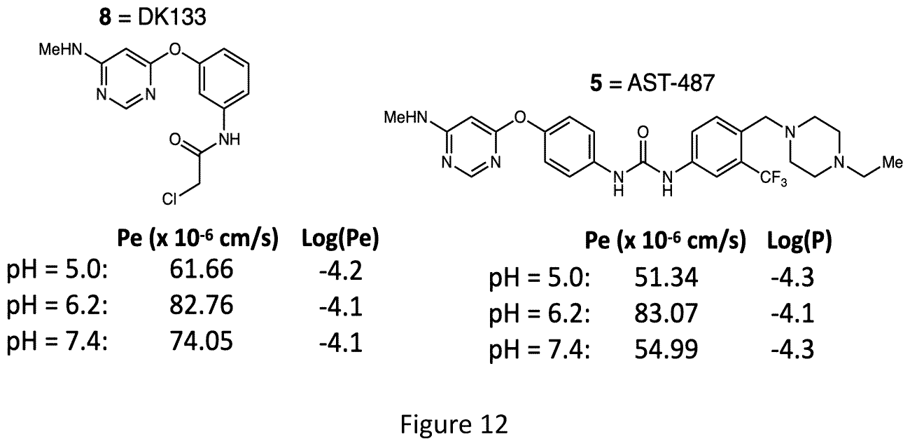

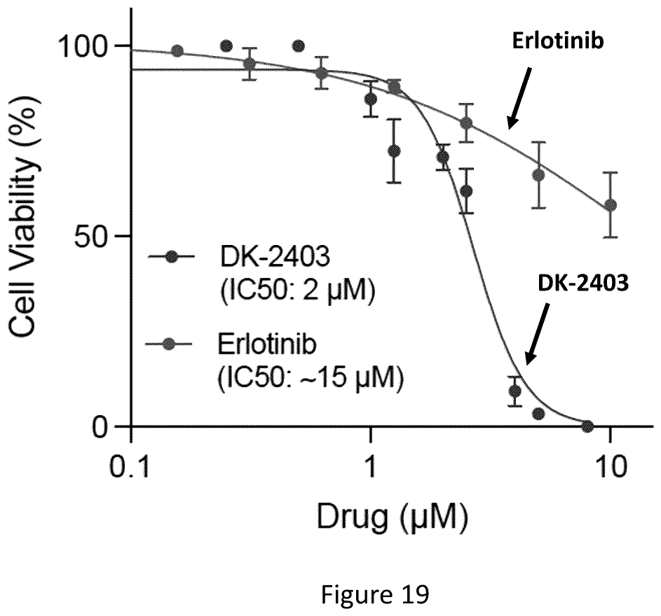

To study the action of DK2403 in living cells, the dose-response cytotoxicity of the compound was assessed in a variety of T-ALL cell lines at 48 hours.⁵¹ As shown in FIG. 8A, 25 displayed marked cytotoxicity, surpassing the potency of 8 (FIG. 11) and JNK-IN-8.¹¹ This effect was resistant to washout, supporting a covalent mechanism (FIG. 8B). Compound 25 is a cytotoxic MAP2K7 inhibitor towards T-ALL lines of interest. Cytotoxicity was resistant to washout, supporting covalency. EGFR inhibitor erlotinib⁵⁵ failed to induce cytotoxicity at similar concentrations, suggesting that the effect on cell viability is likely driven by MAP2K7 inhibition (FIG. 19).

Further probing the cellular efficacy of our MAP2K7 inhibitor, we examined the dose-response effect of 25 on JNK and ATF2 phosphorylation after 24 h of incubation, ensuring no treatment-associated cytotoxicity. Our studies assessed endogenous MAP2K7 inhibition, rather than the inhibition of induced MAP2K7/JNK.³² Treatment with 25 potently attenuated phosphorylation at 5-10 μM (FIG. 8C). Combined with our clean selectivity profile, this finding suggests that cytotoxicity is derived from attenuating aberrant JNK signaling described in these cell lines.^{11,17-18,50}

In conclusion, we have developed potent and selective MAP2K7 inhibitors that covalently engage the unique Cys218 residue within the active site. Lead optimization activities were guided by an iterative cycle of computational modeling, synthesis, and in vitro evaluation. The rapid generation of chemical diversity was enabled by a streamlined one-pot process, much unlike involved synthetic approaches required by compounds exhibiting similar selectivity.⁵⁶⁻⁵⁷ The preliminary selectivity studies described herein support that DK2403 (25) potently inhibits MAP2K7 without significantly disrupting the greater kinome, rendering it an excellent candidate for the study of MAP2K7 in pediatric T-ALL.

### Abbreviations

ALL, acute lymphoblastic leukemia; KLF4, Krüppel-like factor 4; MAP2K7, mitogen-activated protein kinase kinase 7; MEK7, MAPK/Erk kinase 7; JNK, c-Jun N-terminal kinase; 5Z7O, 5Z-7-oxozeaenol; TAK1, transforming growth factor-β (TGF-β)-activated kinase 1; EGFR, epidermal growth factor receptor; FLT3, FMS-like-tyrosine kinase 3; GST, glutathione S-transferase.

### REFERENCES

(1) Hunger, S. P.; Mullighan, C. G. Acute Lymphoblastic Leukemia in Children. N. Engl. J. Med. 2015, 373, 1541-1552.
(2) Pui, C.-H.; Robison, L. L.; Look, A. T. Acute Lymphoblastic Leukaemia. Lancet 2008, 371, 1030-1043.
(3) Pui, C.-H.; Evans, W. E. Treatment of Acute Lymphoblastic Leukemia. N. Engl. J. Med. 2006, 354, 166-178.
(4) Pui, C. H.; Carroll, W. L.; Meshinchi, S.; Arceci, R. J. Biology, Risk Stratification, and Therapy of Pediatric Acute Leukemias: An Update. J. Clin. Oncol. 2011, 29, 551-565.
(5) Linabery, A. M.; Ross, J. A. Trends in Childhood Cancer Incidence in the U.S. (1992-2004). Cancer 2008, 112, 416-432.
(6) Linabery, A. M.; Ross, J. A. Childhood and Adolescent Cancer Survival in the U.S. By Race and Ethnicity (Diagnostic Period 1975-1999). Cancer 2008, 113, 2575-2596.
(7) Ko, R. H.; Ji, L.; Barnette, P.; Bostrom, B.; Hutchinson, R.; Raetz, E.; Seibel, N. L.; Twist, C. J.; Eckroth, E.; Sposto, R.; Gaynon, P. S.; Loh, M. L. Outcome of Patients Treated for Relapsed or Refractory Acute Lymphoblastic Leukemia: A Therapeutic Advances in Childhood Leukemia Consortium Study. J. Clin. Oncol. 2010, 28, 648-654.
(8) Oeffinger, K. C.; Mertens, A. C.; Sklar, C. A.; Kawashima, T.; Hudson, M. M.; Meadows, A. T.; Friedman, D. L.; Marina, N.; Hobbie, W.; Kadan-Lottick, N. S.; Schwartz, C. L.; Leisenring, W.; Robison, L. L. Chronic Health Conditions in Adult Survivors of Childhood Cancer. N. Engl. J. Med. 2006, 355, 1572-1582.
(9) Pinkel, D. Five-Year Follow-up of “Total Therapy” of Childhood Lymphocytic Leukemia. JAMA 1971, 216, 648-652.
(10) Kantarjian, H.; Thomas, D.; Wayne, A. S.; O'Brien, S. Monoclonal Antibody-Based Therapies: A New Dawn in the Treatment of Acute Lymphoblastic Leukemia. J. Clin. Oncol. 2012, 30, 3876-3883.
(11) Shen, Y.; Park, C. S.; Suppipat, K.; Mistretta, T. A.; Puppi, M.; Horton, T. M.; Rabin, K.; Gray, N. S.; Meijerink, J. P. P.; Lacorazza, H. D. Inactivation of Klf4 Promotes T-Cell Acute Lymphoblastic Leukemia and Activates the Map2k7 Pathway. Leukemia 2017, 31, 1314-1324.
(12) Shields, J. M.; Christy, R. J.; Yang, V. W. Identification and Characterization of a Gene Encoding a Gut-Enriched Krüppel-Like Factor Expressed During Growth Arrest. J. Biol. Chem. 1996, 271, 20009-20017.
(13) Black, A. R.; Black, J. D.; Azizkhan-Clifford, J. Sp1 and Krüppel-Like Factor Family of Transcription Factors in Cell Growth, Regulation, and Cancer. J. Cell. Physiol. 2001, 188, 143-160.
(14) Rowland, B. D.; Bernards, R.; Peeper, D. S. The Klf4 Tumour Suppressor Is a Transcriptional Repressor of P53 That Acts as a Context-Dependent Oncogene. Nat. Cell Biol. 2005, 7, 1074-1082.
(15) Kang, H.; Chen, I.-M.; Wilson, C. S.; Bedrick, E. J.; Harvey, R. C.; Atlas, S. R.; Devidas, M.; Mullighan, C. G.; Wang, X.; Murphy, M.; Ar, K.; Wharton, W.; Borowitz, M. J.; Bowman, W. P.; Bhojwani, D.; Carroll, W. L.; Camitta, B. M.; Reaman, G. H.; Smith, M. A.; Downing, J. R.; Hunger, S. P.; Willman, C. L. Gene Expression Classifiers for Relapse-Free Survival and Minimal Residual Disease Improve Risk Classification and Outcome Prediction in Pediatric B-Precursor Acute Lymphoblastic Leukemia. Blood 2010, 115, 1394-1405.
(16) Malik, D.; Kaul, D.; Chauhan, N.; Marwaha, R. K. Mir-2909-Mediated Regulation of Klf4: A Novel Molecular Mechanism for Differentiating between B-Cell and T-Cell Pediatric Acute Lymphoblastic Leukemias. Mol. Cancer 2014, 13, 175.
(17) Lacorazza, H. D. Klf4 Represses Map2k7 Signaling in T-All. Oncotarget 2017, 8, 73366-73367.
(18) Chen, T. J.; Du, W.; Junco, J. J.; Bridges, C. S.; Shen, Y.; Puppi, M.; Rabin, K. R.; Lacorazza, H. D. Inhibition of the Map2k7-Jnk Pathway with 5z-7-Oxozeaenol Induces Apoptosis in T-Cell Acute Lymphoblastic Leukemia. Oncotarget 2021, 12, 1787-1801.
(19) Bridges, C. S.; Chen, T. J.; Puppi, M.; Lacorazza, H. D. Anti-Leukemic Properties of the Kinase Inhibitor Otssp167 in T Cell Acute Lymphoblastic Leukemia. Blood 2021, 138, 3405.
(20) Platanias, L. C. Map Kinase Signaling Pathways and Hematologic Malignancies. Blood 2003, 101, 4667.
(21) Platanias, L. C. Map Kinase Signaling Pathways and Hematologic Malignancies. Blood 2003, 101, 4667-4679.
(22) Park, J. G.; Aziz, N.; Cho, J. Y. Mkk7, the Essential Regulator of Jnk Signaling Involved in Cancer Cell Survival: A Newly Emerging Anticancer Therapeutic Target. Ther. Adv. Med. Oncol. 2019, 11, 1-14.
(23) Van Meter, M.; Simon, M.; Tombline, G.; May, A.; Morello, T. D.; Hubbard, B. P.; Bredbenner, K.; Park, R.; Sinclair, D. A.; Bohr, V. A.; Gorbunova, V.; Seluanov, A. Jnk Phosphorylates Sirt6 to Stimulate DNA Double-Strand Break Repair in Response to Oxidative Stress by Recruiting Parpl to DNA Breaks. Cell Rep. 2016, 16, 2641-2650.
(24) Calses, P. C.; Dhillon, K. K.; Tucker, N.; Chi, Y.; Huang, J. W.; Kawasumi, M.; Nghicm, P.; Wang, Y.; Clurman, B. E.; Jacquemont, C.; Gafken, P. R.; Sugasawa, K.; Saijo, M.; Taniguchi, T. Dgcr8 Mediates Repair of Uv-Induced DNA Damage Independently of Rna Processing. Cell Rep. 2017, 19, 162-174.
(25) Kwong, A. J.; Scheidt, K. A. Non-‘Classical’ Meks: A Review of Mek3-7 Inhibitors. Bioorganic Med. Chem. Lett. 2020, 30, 127203.
(26) Sogabe, Y.; Matsumoto, T.; Hashimoto, T.; Kirii, Y.; Sawa, M.; Kinoshita, T. 5z-7-Oxozeaenol Covalently Binds to Map2k7 at Cys218 in an Unprecedented Manner. Bioorg. Med. Chem. Lett. 2015, 25, 593-596.
(27) Wu, J.; Powell, F.; Larsen, N. A.; Lai, Z.; Byth, K. F.; Read, J.; Gu, R. F.; Roth, M.; Toader, D.; Sach, J. C.; Chen, H. Mechanism and in Vitro Pharmacology of Tak1 Inhibition by (5z)-7-Oxozcaenol. ACS Chem. Biol. 2013, 8, 643-650.
(28) Shraga, A.; Olshvang, E.; Davidzohn, N.; Khoshkenar, P.; Germain, N.; Shurrush, K.; Carvalho, S.; Avram, L.; Albeck, S.; Unger, T.; Lefker, B.; Subramanyam, C.; Hudkins, R. L.; Mitchell, A.; Shulman, Z.; Kinoshita, T.; London, N. Covalent Docking Identifies a Potent and Selective Mkk7 Inhibitor. Cell Chem. Biol. 2019, 26, 98-108.e105.
(29) Wolle, P.; Hardick, J.; Cronin, S. J. F.; Engel, J.; Baumann, M.; Lategahn, J.; Penninger, J. M.; Rauh, D. Targeting the Mkk7-Jnk (Mitogen-Activated Protein Kinase Kinase 7-C-Jun N-Terminal Kinase) Pathway with Covalent Inhibitors. J. Med. Chem. 2019, 62, 2843-2848.
(30) Gehrtz, P.; Marom, S.; Bührmann, M.; Hardick, J.; Kleinbölting, S.; Shraga, A.; Dubiella, C.; Gabizon, R.; Wiese, J. N.; Müller, M. P.; Cohen, G.; Babaev, I.; Shurrush, K.; Avram, L.; Resnick, E.; Barr, H.; Rauh, D.; London, N. Optimization of Covalent Mkk7 Inhibitors Via Crude Nanomole-Scale Libraries. J. Med. Chem. 2022, 65, 10341-10356.
(31) Jiang, J.; Jiang, B.; He, Z.; Ficarro, S. B.; Che, J.; Marto, J. A.; Gao, Y.; Zhang, T.; Gray, N. S. Discovery of Covalent Mkk4/7 Dual Inhibitor. Cell Chem. Biol. 2020, 27, 1553-1560.e1558.
(32) Schröder, M.; Tan, L.; Wang, J.; Liang, Y.; Gray, N. S.; Knapp, S.; Chaikuad, A. Catalytic Domain Plasticity of Mkk7 Reveals Structural Mechanisms of Allosteric Activation and Diverse Targeting Opportunities. Cell Chem. Biol. 2020, 27, 1285-1295.e1284.
(33) Deibler, K. K.; Mishra, R. K.; Clutter, M. R.; Antanasijevic, A.; Bergan, R.; Caffrey, M.; Scheidt, K. A. A Chemical Probe Strategy for Interrogating Inhibitor Selectivity across the Mek Kinase Family. ACS Chem. Biol. 2017, 12, 1245-1256.
(34) Worzella, T.; Butzler, M.; Hennek, J.; Hanson, S.; Simdon, L.; Goueli, S.; Cowan, C.; Zegzouti, H. A Flexible Workflow for Automated Bioluminescent Kinase Selectivity Profiling. SLAS Technol. 2017, 22, 153-162.
(35) Shieh, W.-C.; McKenna, J.; Sclafani, J. A.; Xue, S.; Girgis, M.; Vivelo, J.; Radetich, B.; Prasad, K. Syntheses of a Triad of Flt3 Kinase Inhibitors: From Bench to Pilot Plant. Org. Process Res. Dev. 2008, 12, 1146-1155.
(36) Weisberg, E.; Roesel, J.; Bold, G.; Furet, P.; Jiang, J.; Cools, J.; Wright, R. D.; Nelson, E.; Barrett, R.; Ray, A. Antileukemic Effects of the Novel, Mutant Flt3 Inhibitor Nvp-Ast487: Effects on Pkc412-Sensitive and -Resistant Flt3-Expressing Cells. Blood 2008, 112, 5161-5170.
(37) Akeno-Stuart, N.; Croyle, M.; Knauf, J. A.; Malaguarnera, R.; Vitagliano, D.; Santoro, M.; Stephan, C.; Grosios, K.; Wartmann, M.; Cozens, R.; Caravatti, G.; Fabbro, D.; Lane, H. A.; Fagin, J. A. The Ret Kinase Inhibitor Nvp-Ast487 Blocks Growth and Calcitonin Gene Expression through Distinct Mechanisms in Medullary Thyroid Cancer Cells. Cancer Res. 2007, 67, 6956-6964.
(38) Liu, Q.; Sabnis, Y.; Zhao, Z.; Zhang, T.; Buhrlage, Sara J.; Jones, Lyn H.; Gray, Nathanael S. Developing Irreversible Inhibitors of the Protein Kinase Cysteinome. Chem. Biol. 2013, 20, 146-159.
(39) Kufareva, I.; Abagyan, R. Type-Ii Kinase Inhibitor Docking, Screening, and Profiling Using Modified Structures of Active Kinase States. J. Med. Chem. 2008, 51, 7921-7932.
(40) Wells, C. I.; Vasta, J. D.; Corona, C. R.; Wilkinson, J.; Zimprich, C. A.; Ingold, M. R.; Pickett, J. E.; Drewry, D. H.; Pugh, K. M.; Schwinn, M. K.; Hwang, B.; Zegzouti, H.; Huber, K. V. M.; Cong, M.; Meisenheimer, P. L.; Willson, T. M.; Robers, M. B. Quantifying Cdk Inhibitor Selectivity in Live Cells. Nat. Commun. 2020, 17, 2743.
(41) Gehringer, M.; Laufer, S. A. Emerging and Re-Emerging Warheads for Targeted Covalent Inhibitors: Applications in Medicinal Chemistry and Chemical Biology. J. Med. Chem. 2019, 62, 5673-5724.
(42) Flanagan, M. E.; Abramite, J. A.; Anderson, D. P.; Aulabaugh, A.; Dahal, U. P.; Gilbert, A. M.; Li, C.; Montgomery, J.; Oppenheimer, S. R.; Ryder, T.; Schuff, B. P.; Uccello, D. P.; Walker, G. S.; Wu, Y.; Brown, M. F.; Chen, J. M.; Hayward, M. M.; Noc, M. C.; Obach, R. S.; Philippe, L.; Shanmugasundaram, V.; Shapiro, M. J.; Starr, J.; Stroh, J.; Che, Y. Chemical and Computational Methods for the Characterization of Covalent Reactive Groups for the Prospective Design of Irreversible Inhibitors. J. Med. Chem. 2014, 57, 10072-10079.
(43) Lu, W.; Kostic, M.; Zhang, T.; Che, J.; Patricelli, M. P.; Jones, L. H.; Chouchani, E. T.; Gray, N. S. Fragment-Based Covalent Ligand Discovery. RSC Chem. Biol. 2021, 2, 354-367.
(44) Douangamath, A.; Fearon, D.; Gehrtz, P.; Krojer, T.; Lukacik, P.; Owen, C. D.; Resnick, E.; Strain-Damerell, C.; Aimon, A.; Ábrányi-Balogh, P.; Brandão-Neto, J.; Carbery, A.; Davison, G.; Dias, A.; Downes, T. D.; Dunnett, L.; Fairhead, M.; Firth, J. D.; Jones, S. P.; Keeley, A.; Keserü, G. M.; Klein, H. F.; Martin, M. P.; Noble, M. E. M.; O'Brien, P.; Powell, A.; Reddi, R. N.; Skyner, R.; Snec, M.; Waring, M. J.; Wild, C.; London, N.; von Delft, F.; Walsh, M. A. Crystallographic and Electrophilic Fragment Screening of the Sars-Cov-2 Main Protease. Nat. Commun. 2020, 11, 5047.
(45) Resnick, E.; Bradley, A.; Gan, J.; Douangamath, A.; Krojer, T.; Sethi, R.; Geurink, P. P.; Aimon, A.; Amitai, G.; Bellini, D.; Bennett, J.; Fairhead, M.; Fedorov, O.; Gabizon, R.; Gan, J.; Guo, J.; Plotnikov, A.; Reznik, N.; Ruda, G. F.; Díaz-Sáez, L.; Straub, V. M.; Szommer, T.; Velupillai, S.; Zaidman, D.; Zhang, Y.; Coker, A. R.; Dowson, C. G.; Barr, H. M.; Wang, C.; Huber, K. V. M.; Brennan, P. E.; Ovaa, H.; von Delft, F.; London, N. Rapid Covalent-Probe Discovery by Electrophile-Fragment Screening. J. Am. Chem. Soc. 2019, 141, 8951-8968.
(46) Schröder, M.; Chaikuad, A. Assessing Reversible and Irreversible Binding Effects of Kinase Covalent Inhibitors through Adp-Glo Assays. STAR Protocols 2021, 2, 100717.
(47) Kwong, A. J.; Pham, T. N. D.; Oelschlager, H. E.; Munshi, H. G.; Scheidt, K. A. Rational Design, Optimization, and Biological Evaluation of Novel Mek4 Inhibitors against Pancreatic Adenocarcinoma. ACS Med. Chem. Lett. 2021, 12, 1559-1567.
(48) Deibler, K. K.; Schiltz, G. E.; Clutter, M. R.; Mishra, R. K.; Vagadia, P. P.; O'Connor, M.; George, M. D.; Gordon, R.; Fowler, G.; Bergan, R.; Scheidt, K. A. Synthesis and Biological Evaluation of 3-Arylindazoles as Selective Mek4 Inhibitors. ChemMedChem 2019, 14, 615-620.
(49) Karaman, M. W.; Herrgard, S.; Treiber, D. K.; Gallant, P.; Atteridge, C. E.; Campbell, B. T.; Chan, K. W.; Ciceri, P.; Davis, M. I.; Edeen, P. T.; Faraoni, R.; Floyd, M.; Hunt, J. P.; Lockhart, D. J.; Milanov, Z. V.; Morrison, M. J.; Pallares, G.; Patel, H. K.; Pritchard, S.; Wodicka, L. M.; Zarrinkar, P. P. A Quantitative Analysis of Kinase Inhibitor Selectivity. Nat. Biotechnol. 2008, 26, 127-132.
(50) Shen, Y.; Chen, T. J.; Lacorazza, H. D. Novel Tumor-Suppressor Function of Klf4 in Pediatric T-Cell Acute Lymphoblastic Leukemia. Exp. Hematol. 2017, 53, 16-25.
(51) Zhang, T.; Hatcher, J. M.; Teng, M.; Gray, N. S.; Kostic, M. Recent Advances in Selective and Irreversible Covalent Ligand Development and Validation. Cell Chem. Biol. 2019, 26, 1486-1500.
(52) Bridges, C. S.; Chen, T. J.; Puppi, M.; Rabin, K. R.; Lacorazza, H. D. Anti-Leukemic Properties of the Kinase Inhibitor Otssp167 in T Cell Acute Lymphoblastic Leukemia. Blood Advances 2022, bloodadvances.2022008548.
(53) Ellestad, G. A. (52)-7-Oxozcaenol: A Novel and Potent Resorcylic Acid Lactone Kinase Inhibitor with a Cis-Enone Michael Acceptor. Chirality 2019, 37, 110-117.
(54) Klaeger, S.; Heinzlmeir, S.; Wilhelm, M.; Polzer, H.; Vick, B.; Kocnig, P.-A.; Reinecke, M.; Ruprecht, B.; Petzoldt, S.; Meng, C.; Zecha, J.; Reiter, K.; Qiao, H.; Helm, D.; Koch, H.; Schoof, M.; Canevari, G.; Casale, E.; Depaolini Stefania, R.; Feuchtinger, A.; Wu, Z.; Schmidt, T.; Rueckert, L.; Becker, W.; Huenges, J.; Garz, A.-K.; Gohlke, B.-O.; Zolg Daniel, P.; Kayser, G.; Vooder, T.; Preissner, R.; Hahne, H.; Tonisson, N.; Kramer, K.; Götze, K.; Bassermann, F.; Schlegl, J.; Ehrlich, H.-C.; Aiche, S.; Walch, A.; Greif Philipp, A.; Schneider, S.; Felder Eduard, R.; Ruland, J.; Médard, G.; Jeremias, I.; Spiekermann, K.; Kuster, B. The Target Landscape of Clinical Kinase Drugs. Science 2017, 358, eaan4368.
(55) Moyer, J. D.; Barbacci, E. G.; Iwata, K. K.; Arnold, L.; Boman, B.; Cunningham, A.; DiOrio, C.; Doty, J.; Morin, M. J.; Moyer, M. P.; Neveu, M.; Pollack, V. A.; Pustilnik, L. R.; Reynolds, M. M.; Sloan, D.; Theleman, A.; Miller, P. Induction of Apoptosis and Cell Cycle Arrest by Cp-358,774, an Inhibitor of Epidermal Growth Factor Receptor Tyrosine Kinase. Cancer Res. 1997, 57, 4838-4848.
(56) Chandregowda, V.; Venkateswara Rao, G.; Chandrasekara Reddy, G. Convergent Approach for Commercial Synthesis of Gefitinib and Erlotinib. Org. Process Res. Dev. 2007, 11, 813-816.
(57) Kompella, A.; Adibhatla, B. R. K.; Muddasani, P. R.; Rachakonda, S.; Gampa, V. K.; Dubey, P. K. A Facile Total Synthesis for Large-Scale Production of Imatinib Base. Org. Process Res. Dev. 2012, 16, 1794-1804.

### Experimental

### ScanEDGE Assay

The ScanEDGE assay, completed by DiscoverX (Eurofins) is a proprietary active site-directed competition binding assay. Compounds that bind the kinase active site prevent kinase binding to the immobilized ligand, reducing the amount of kinase captured on the solid support. Hits are identified by measuring the amount of kinase captured in experimental vs, control samples by a qPCR method that detects an associated DNA label. According to data provided by Eurofins, a typical hit at 5-10% of control signal in a 1 μM assay as shown above is generally associated with a K_{D} in the range of 100-1,000 nM. Readings with greater than 35% of control signal correspond to exceeding large dissociation constants.

### Selectivity Score

Selectivity score (or S-score) is a quantitative measure of compound selectivity corresponding to the ratio of hits to the number of screened wildtype kinases.

Upon adding additional testing at MEK3, MEK5, and MEK6, the selectivity score of DK2403 is still represented as 1/93=0.011. See Table 1 below.

TABLE 1
S-score Table for LOB002-01-p-0001.

| | | | Number | Screening | |
|---|---|---|---|---|---|
| | Selectivity | | of Non- | Concen- | |
| Compound | Score | Number | Mutant | tration | Selectivity |
| Name | Type | of Hits | Kinases | (nM) | Score |
| DK2403 | S(35) | 1 | 90 | 1000 | 0.011 |
| DK2403 | S(10) | 1 | 90 | 1000 | 0.011 |
| DK2403 | S(1) | 0 | 90 | 1000 | 0 |

### ADP-Glo Kinase Activity Assay

Procedure for a 5 μL Kinase reaction using the ADP-Glo Kinase Assay Kit from Promega. Staurosporine was used as the positive control, and DMSO (vehicle) was employed as the negative control. Activity of recombinant active full-length human MEK7 (Carna Biosciences) was measured with recombinant full-length human substrate JNK1 (MAPK8). Using a Mantis, a mixture of MEK7 and JNK1 was dispensed into a 384-well low volume white Proxiplate. For each well, 4 ng of MEK7 and 600 ng of JNK1 in kinase buffer (CST) were used, accounting for 1 μL of this mixture per well. Inhibitor was prepared in a DMSO solution (40 mM). A dose-response dilution was performed in a 384-well Echo transfer plate. Using an Echo Access, 10 nL of each solution was dispensed into the same 384-well low volume white Proxiplate as above. This mixture was allowed to incubate for 30 minutes at room temperature. Using a Mantis, 1 uL of an 8 μM ATP solution was added to each well, and the plate was allowed to incubate for another 1 hour at room temperature. Using the Mantis again, 4 μL of ADP Glo Reagent was dispensed into each well, and the plate was allowed to incubate for 45 min at room temperature. Finally, 9 μL of Kinase Detection Reagent was added manually to each well, and the plate was allowed to incubate for 45 minutes at room temperature. Luminescence was read on a Synergy Neo2 Plate Reader with filter cubes 3 and 114. Integration time was set to 0.20 seconds, gain set to 160, and read height set at 6 mm. All experiments were performed in triplicate on the plate, and at least in one separate technical duplicate was performed. Each experiment's IC₅₀ values are reported as (IC₅₀±SEM) and are provided under each compound's characterization data in the tabulated data section below.

MEK4 selectivity was performed in the same manner with activated MEK4 (Carna Biosciences). Here, p-38α was employed as the kinase substrate in lieu of JNK1 due to the availability of reagents. In addition to staurosporine, MEK4 inhibitor AJK339 (IC₅₀=61 nM, ACS Med. Chem. Lett. 2021, 12, 10, 1559) was employed as a positive control (see FIG. 9). To probe the ATP-competitive nature of 25, this compound was subjected to a similar assay forgoing the 30 minute incubation time described above (STAR Protocols 2021, 2, 100717). The results are tabulated below in compound characterization. Potency determined by duplicate of triplicates corresponded to a IC₅₀ value, which is in line with the ratio difference observed among other covalent inhibitors (for examples, see Cell Chem. Biol. 2020, 27, 1285).

### Computational Methods

Covalent docking of analogs was carried out with the structure of MEK7 (PDB: 6YG3) using the Schrodinger Maestro platform. The protein was prepared with the Protein Preparation tool to add and orient missing hydrogens, set protonation state to physiologic pH, and subject the structure to energy minimization using an OPLS3e force field. Ligands were prepared using the LigPrep tool to set protonation states to pH 7.4±1.0 and to generate all possible chiral configurations. Finally, ligands were docked with CovDock. Cys218 was identified as the reactive residue, and a 10 Å×10 Å×10 Å box centered on the centroid of the co-crystallized ligand was used to define the binding site for ligand docking. The reaction type was set to nucleophilic substitution with no other constraints applied, and the docking mode was set to pose prediction. Poses were visualized in Maestro and used for figures in this publication.

### Whole-Protein LC/TOF-MS

Inactive MAP2K7 was acquired from Carna Biosciences (product number: 07-147-10). The construct was full-length MAP2K7 (1-419 amino acids of accession number NP_660186.1), which was prepared by the manufacturer as an N-terminal GST-fusion protein using an E. coli expression system. The construct was then purified by glutathione sepharose chromatography and shipped in a storage buffer containing 50 mM Tris —HCl, 150 mM NaCl, 0.05 mM Brij-35, 1 mM DTT, and 10% glycerol at pH=7.5.

Each whole-protein MS experiment was run with a 10 uL injection of a sample containing 50 μg of protein diluted to a final volume of 100 μL. The construct aliquots were first diluted to the appropriate volume with an aqueous solution of 150 mM NaCl and 50 mM Tris —HCl. The diluted samples were loaded on a Pierce spin column (ThermoFisher Scientific catalog number: 87777) to remove Brij-35 and finally eluted with the appropriate volume of the same buffer. Finally, 1.3 uL of DMSO (vehicle) or a 1 mM DMSO solution of covalent inhibitor was added, and the resulting roughly 101 uL solutions were incubated at 5° C. for 12 hours. The incubates were injected (10 uL) on an Agilent 6201 MSLC-TOF (ESI) with a protein column. A 10 minute water:acetonitrile gradient was performed, eluting recombinant proteins of interest in roughly 5 minutes. Masses were deconvoluted using MassHunter BioConfirm (maximum entropy).

### LC/MS-MS Determination of Residue Selectivity

MAP2K7 recombinant protein was diluted, purified, and incubated as described above. After incubation, protein solution was denatured with 8 M urea for 10 minutes at 65° C., reduced with 12.5 mM DTT for 15 minutes at 65° C., and alkylated with 25 mM iodoacetamide in the absence of light for 30 minutes at 37° C. Protein solution was diluted with PBS to 2 M urea and divided into two digestion reactions: one with 2 μg trypsin and 10 μg chymotrypsin for 6 hours at 37° C., the other with 5 μg GluC for 6 hours at 37° C. Digested peptides were acidified with 5% formic acid and desalted using Sep-Pak C18 1 cc Vac Cartridge (Waters). Peptides were analyzed by liquid chromatography tandem mass-spectrometry using an Orbitrap Eclipse Tribrid Mass Spectrometer (Thermo Scientific) coupled to an Vanquish Neo UHPLC System (Thermo Scientific). Peptides were injected onto EASY-Spray HPLC Column (C18, 2 μm particle size, 75 μm inner diameter, 250 mm length) and separated at a flow rate of 0.25 L/min using the following gradient: 5% buffer B (80% acetonitrile with 0.1% formic acid) in buffer A (water with 0.1% formic acid) from 0-15 min, 5-45% buffer B from 15-155 min, 45-100% buffer B from 155-180 min. Raw data was searched in Proteome Discoverer 2.5 (Thermo Scientific).

### T-ALL Cell Lines

T-ALL cell lines were cultured in RPMI-1640 medium supplemented with 10% fetal bovine serum: JURKAT, KOPT-K1, RPMI-8402, and ALL-SIL. Cells were tested for mycoplasma and authenticated using STR fingerprinting every six months.

### Cytotoxicity Assays

Cell lines were plated in triplicates at a cell density of 2×10⁴ cells per well (96-well plate) and cultured for 48 hours in the presence of MAP2K7 inhibitor or vehicle control (DMSO). Cell viability was measured using CellTiter-Glo Luminescent cell viability assay. The half-maximal inhibitory concentration (IC₅₀) was calcd by nonlinear regression analysis using GraphPad software.

### Washout Experiments

T-cell cell lines Jurkat and KOPTK1 were plated in triplicates (20×10⁴ cells per 96-well plate) and cultured with different concentrations of DK-2403 compound for 18 hours. Cells were washed twice to remove DK-2403 and incubated for 30 hours. Cell viability was measured (48 hours) using CellTiter-Glo luminescent cell viability assay. The half-maximal inhibitory concentration (IC₅₀) was calculated using nonlinear regression analysis via GraphPad software.

### Immunoblot Analysis

Cells were lysed with SDS lysis buffer containing (10 mM Tris pH 7.4 containing 1% SDS and 1 mM PMSF) and supplemented with Halt Protease and Phosphatase Inhibitor Cocktail (ThermoFisher) after 24 h of incubation. Protein lysates were electrophoresed onto SDS PAGE gel and transferred to the PVDF membrane using the iBLOT system. Antibodies phospho-SAPK/JNK (clone 81E11) (#4668) and SAPK/JNK (#9252) were used at 1:5000 dilution. Secondary antibodies cross-linked with HRP (anti-rabbit IgG #7074 and anti-mouse IgG #7076) were used for respective primary antibodies at concentrations 1:20,000. Protein detection was performed using West Femto Maximum Sensitivity Substrate (ThermoFisher) and Amersham Hyperfilm ECL (GE).

### Chemistry

### General Information

All reactions were carried out under an argon or nitrogen atmosphere in flame-dried glassware with magnetic stirring. All solvents were purified by passing through a bed of activated alumina. Acid chlorides were purified by distillation immediately prior to use. DIPEA was purified by passing through a bed of activated alumina and dried over 3 Å MS. Purification of reaction products was carried out by flash chromatography on Biotage Isolera 4 systems with ultra-grade silica cartridges. Analytical thin layer chromatography was performed on 0.25 mm silica gel 60-F plates. Visualization was accomplished with UV light. ¹H NMR spectra were recorded on a Bruker AVANCE III 500 MHZ w/DCH Cryoprobe (500 MHZ) spectrometer and are reported in ppm using solvent as an internal standard (CDCl₃ at 7.26 ppm, DMSO at 2.50 ppm, CD₃OD at 3.31 ppm). Data are reported as (ap=apparent, s=singlet, d=doublet, t=apparent triplet, q=quartet, m=multiplet, b=broad; coupling constant(s) in Hz; integration) Proton-decoupled ¹³C NMR spectra were recorded on a Bruker AVANCE III 500 MHZ w/DCH Cryoprobe (126 MHz) spectrometer and are reported in ppm using residual solvent as an internal standard (CDCl₃ at 77.16 ppm, DMSO at 39.52 ppm, CD₃OD at 49.0 ppm). ¹⁹F NMR spectra were recorded on a Bruker AVANCE III HD 500 MHz w/BBO Prodigy Probe. Mass spectra were obtained on a WATERS Acquity I-Class UPLC-MS with a single quad detector (ESI), ELSD, and PDA. Compounds submitted to biological testing were found to be >90% pure, assessed by ¹H NMR (AVANCE III 500 MHz w/direct cryoprobe (500 MHz) spectrometer) and UHPLC-MS (WATERS Acquity-H UPLC-MS with a SQD). High-resolution mass spectrometry (HRMS) was obtained using an Agilent 6201 MSLC-TOF (ESI). FTIR data was collected at room temperature on a Bruker Tensor 37 FTIR Spectrometer equipped with a Mid IR detector and KBr beam splitter in attenuated total reflectance (ATR) mode in the range of 4000 to 600 cm-1, averaged over 16 scans. The OPUS software was used for the data acquisition.

### One-Pot Procedure Development

Towards the validation of the one-pot procedures described below, the intermediates (SI-1 and SI-2) were isolated stepwise and characterized. Please see the experimental procedures and characterization below.

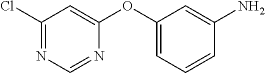 [SMILES: Nc1cccc(Oc2cc(Cl)ncn2)c1]

3-((6-chloropyrimidin-4-yl)oxy) aniline (SI-1). To a flame-dried vial was added 3-aminophenol, which was suspended in DMF (0.5 M). The solution was cooled to 0° C., and sodium hydride (1.1 equiv, 60 wt %) was added portion-wise. The reaction was allowed to stir and warm to room temperature. After 30 min, the reaction mixture was heated to 80° C., and 4,6-dichloropyrimidine (1.0 equiv) was added as a solid. The reaction was determined to be complete by UHPLC-MS analysis after a period of 30 min. The reaction mixture was diluted with deionized water (10 volumes) and extracted with ethyl acetate (3×1 volume). The combined organic extracts were dried over anhydrous Na₂SO₄, filtered through glass wool, and concentrated in vacuo. The product was purified by flash column chromatography (SiO2, 1:1 hexanes:acetone) to afford SI-1 (210 mg, 94%) as a colorless oil.

### Analytical Data for SI-1:

¹H NMR (500 MHZ, CDCl₃) δ 8.65 (s, 1H), 7.24 (ap t, J=8.1 Hz, 1H), 6.91 (ap d, J=1.0 Hz, 1H), 6.64 (ap dd, J=8.0, 0.9 Hz, 1H), 6.54 (ap dd, J=8.0, 0.9 Hz, 1H), 6.48 (ap t, J=2.2 Hz, 1H), 3.90 (br s, 2H).

¹³C NMR (126 MHz, CDCl₃) δ 170.56, 161.96, 158.66, 152.96, 148.51, 130.67, 113.05, 110.69, 107.68, 107.57.

LRMS (ESI): Exact mass calcd for C₁₀H₉ClN₃O⁺ [M+H]⁺, 222.0. Found 222.1.

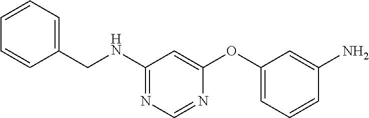 [SMILES: Nc1cccc(Oc2cc(NCc3ccccc3)ncn2)c1]

6-(3-aminophenoxy)-N-benzylpyrimidin-4-amine (SI-2). To a flame-dried vial was added 3-((6-chloropyrimidin-4-yl)oxy) aniline (SI-1), which was suspended in DMF (0.5 M). Next, benzylamine (1.0 equiv) and DIPEA (1.1 equiv) were added, and the resultant solution was heated to 80° C. After a period of 4 hours, UHPLC indicated the consumption of both starting materials. The reaction mixture was diluted with deionized water (10 volumes) and extracted with ethyl acetate (3×1 volume). The combined organic extracts were dried over anhydrous Na₂SO₄, filtered through glass wool, and concentrated in vacuo. The solid residue was suspended in CH₂Cl₂ and filtered, and the collected solid was washed with diethyl ether. Removal of residual solvent in vacuo afforded SI-2 (190 mg, 88%) as an off-white solid.

### Analytical Data for SI-2:

¹H NMR (500 MHz, DMSO) δ 8.20 (s, 1H), 7.88 (s, 1H), 7.36-7.28 (m, 4H), 7.24 (m, 1H), 7.06 (ap t, J=8.0 Hz, 1H), 6.48 (ap dd, J=8.1, 2.2 Hz, 1H), 6.35 (ap d, J=2.7 Hz, 1H), 6.31-6.20 (m, 1H), 5.84 (s, 1H), 5.29 (s, 2H), 4.53 (s, 2H).

¹³C NMR (126 MHz, DMSO) δ 169.85, 165.07, 158.69, 154.28, 150.88, 139.99, 130.40, 128.81, 127.70, 127.29, 111.38, 108.63, 106.91, 87.46, 44.22.

LRMS (ESI): Exact mass calcd for C₁₇H₁₇N₄O+ [M+H]⁺, 293.1. Found 293.0.

### General Procedures

### General Procedure A

To a flame-dried vial was added 3-aminophenol, which was suspended in DMF (0.5 M). The solution was cooled to 0° C., and sodium hydride (1.1 equiv, 60 wt %) was added portion-wise. The reaction was allowed to stir and warm to room temperature. After 30 min, the reaction mixture was heated to 80° C., and 4,6-dichloropyrimidine (1.05 equiv) was added as a solid. The resultant mixture was allowed to stir at 80° C. for roughly 5 hours while the reaction was monitored by UHPLC-MS. Upon full conversion of starting material, a preponderance of SI-1 was observed by UHPLC-MS, and the amine nucleophile was added (1.0 equiv), followed by DIPEA (2.1 equiv). Following full consumption of the amine, the reaction mixture was again cooled to 0° C. Acyl chloride (1.4 equiv) was then added dropwise, and the reaction was allowed to stir and warm to room temperature. Finally, the reaction was diluted with water (10 volumes) and extracted with ethyl acetate (3×1 volume). The combined organic extracts were dried over anhydrous Na₂SO₄, filtered through glass wool, and concentrated in vacuo. The product was purified by silica gel column chromatography, eluting with roughly 2:1 hexanes:acetone.

### General Procedure B

To a flame-dried vial was added 4-aminophenol, which was suspended in DMF (0.5 M). The solution was cooled to 0° C., and sodium hydride (1.1 equiv, 60 wt %) was added portion-wise. The reaction was allowed to stir and warm to room temperature. After 30 min, the reaction mixture was heated to 80° C., and 4,6-dichloropyrimidine (1.0 equiv) was added as a solid. The resultant mixture was allowed to stir at 80° C. for roughly 5 hours while the reaction was monitored by UHPLC-MS. Upon full conversion of starting material, the reaction mixture was cooled to 30° C., and the amine nucleophile was added (5.0 equiv), followed by DIPEA (2.1 equiv). Following full consumption of the amine, the reaction mixture was again cooled to 0° C. Acyl chloride (1.4 equiv) was then added dropwise, and the reaction was allowed to stir and warm to room temperature. Finally, the reaction was diluted with water (10 volumes) and extracted with ethyl acetate (3×1 volume). The combined organic extracts were dried over anhydrous Na₂SO₄, filtered through glass wool, and concentrated in vacuo. The product was purified by silica gel column chromatography, eluting with roughly 2:1 hexanes:acetone.

### Synthesis and Characterization of Notable Compounds

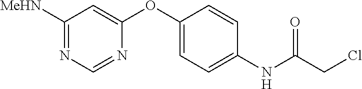 [SMILES: CNc1cc(Oc2ccc(NC(=O)CCl)cc2)ncn1]

2-chloro-N-(4-((6-(methylamino)pyrimidin-4-yl)oxy)phenyl)acetamide (6). Prepared according to general procedure B using methylamine (33 wt. % in EtOH) and chloroacetyl chloride, affording 6 as a white solid (21 mg, 27%).

### Analytical Data for 6:

FTIR (diamond, anvil, solid) cm⁻¹:3281.59, 1597.57, 1558.47, 1540.90, 1437.52, 1259.74, 1193.97, 1022.82, 985.76, 668.08.

¹H NMR (500 MHz, DMSO) δ 10.49 (s, 1H), 8.15 (s, 1H), 7.46 (ap d, J=2.7 Hz, 1H), 7.44-7.31 (m, 3H), 6.87 (ap d, J=2.3 Hz, 1H), 5.81 (s, 1H), 4.26 (s, 2H), 2.78 (s, 3H)

¹³C NMR (126 MHz, DMSO) δ 165.30, 158.61, 153.67, 140.20, 130.43, 117.12, 116.20, 112.64, 87.77, 44.02, 27.95.

HRMS (ESI): Exact mass calcd for C₁₃H₁₄ClN₄O₂⁺ [M+H]⁺, 293.0800. Found 293.0804.

MEK7 IC₅₀: 1.8 μM [1.5±0.21, n=3; 2.1±1.1, n=3].

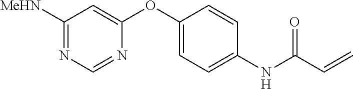 [SMILES: C=CC(=O)Nc1ccc(Oc2cc(NC)ncn2)cc1]

N-(4-((6-(methylamino)pyrimidin-4-yl)oxy)phenyl) acrylamide (7). Prepared according to general procedure B using methylamine (33 wt. % in EtOH) and acryloyl chloride, affording 7 as a yellowish solid (15 mg, 14%).

### Analytical Data for 7:

FTIR (diamond, anvil, solid) cm⁻¹:3289.15, 1654.03, 1590.04, 1544.33, 1259.74, 1098.81, 858.54, 667.09.

¹H NMR (500 MHZ, DMSO) δ 10.26 (s, 1H), 8.14 (s, 1H), 7.54 (s, 1H), 7.46 (ap d, J=8.3 Hz, 1H), 7.40-7.30 (m, 2H), 6.87-6.80 (m, 1H), 6.42 (dd, J=17.0, 10.1 Hz, 1H), 6.26 (dd, J=17.0, 2.0 Hz, 1H), 5.84-5.75 (m, 2H), 2.78 (s, 3H).

¹³C NMR (126 MHZ, CDCl₃) δ 165.46, 163.39, 158.27, 144.93, 143.95, 135.02, 130.99, 128.03, 122.39, 122.13, 85.29, 28.47.

HRMS (ESI): Exact mass calcd for C₁₄H₁₅N₄O₂⁺ [M+H]⁺, 271.1190. Found 271.1191.

MEK7 IC₅₀: 8.8 UM [7.7±1.3, n=3; 9.9±1.2, n=3].

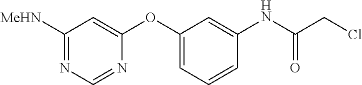 [SMILES: CNc1cc(Oc2cccc(NC(=O)CCl)c2)ncn1]

2-chloro-N-(3-((6-(methylamino)pyrimidin-4-yl)oxy)phenyl)acetamide (8=DK133). Prepared according to modified general procedure A using methylamine (5 equiv, 33 wt. % in EtOH, stirred at 30° C.) and chloroacetyl chloride, affording 8 as a pale yellow amorphous solid (33 mg, 18%).

### Analytical Data for 8:

FTIR (diamond, anvil, solid) cm⁻¹:3313.47, 1653.23, 1598.34, 1557.72, 1438.95, 1259.30, 1195.23, 1016.92, 795.38, 686.49, 668.17.

¹H NMR (500 MHZ, DMSO) δ 10.42 (s, 1H), 8.14 (s, 1H), 7.44 (ap t, J=2.1 Hz, 1H), 7.41-7.30 (m, 3H), 6.89-6.83 (m, 1H), 5.81 (s, 1H), 4.25 (s, 2H), 2.77 (s, 3H).

¹³C NMR (126 MHz, CDCl₃) δ 181.07, 165.26, 163.98, 157.89, 153.37, 138.20, 130.08, 118.07, 116.83, 113.44, 85.86, 42.96, 28.46.

HRMS (ESI): Exact mass calcd for C₁₃H₁₄ClN₄O₂⁺ [M+H]⁺, 293.0800. Found 293.0798

MEK7 IC₅₀: 0.060 μM [0.087±0.041, n=3; 0.034±0.042, n=3].

MEK4 IC₅₀: >80 μM.

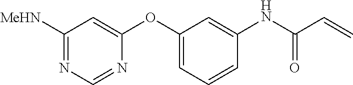 [SMILES: C=CC(=O)Nc1cccc(Oc2cc(NC)ncn2)c1]

N-(3-((6-(methylamino)pyrimidin-4-yl)oxy)phenyl) acrylamide (9). Prepared according to modified general procedure A using methylamine (5 equiv, 33 wt. % in EtOH, stirred at 30° C.) and acryloyl chloride, isolated as a white solid (27 mg, 31%).

### Analytical Data for 9:

FTIR (diamond, anvil, solid) cm⁻¹:3275.12, 1596.02, 1550.40, 1437.10, 1400.50, 1258.76, 1194.35, 1021.25, 984.04, 795.29, 688.46.

¹H NMR (500 MHZ, CD₃OD) δ 8.15 (s, 1H), 7.63 (ap t, J=2.2 Hz, 1H), 7.50-7.45 (m, 1H), 7.40 (ap t, J=8.1 Hz, 1H), 6.93-6.86 (m, 1H), 6.48-6.33 (m, 2H), 5.79 (dd, J=9.6, 2.2 Hz, 1H), 5.76 (s, 1H), 2.86 (s, 3H) [protic N—H signals not observed].

¹³C NMR (126 MHz, CD₃OD) δ 169.46, 165.51, 164.74, 157.59, 153.27, 140.10, 130.86, 129.84, 126.90, 116.59, 116.54, 116.43, 112.69, 87.37.

HRMS (ESI): Exact mass calcd for C₁₄H₁₅N₄O₂⁺ [M+H]⁺, 271.1190. Found 271.1194.

MEK7 IC₅₀: 0.42μ M [0.23±0.19, n=3; 0.61±0.40, n=3].

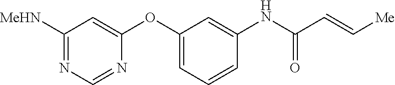 [SMILES: C/C=C/C(=O)Nc1cccc(Oc2cc(NC)ncn2)c1]

(E/Z)—N-(3-((6-(methylamino)pyrimidin-4-yl)oxy)phenyl) but-2-enamide (10). Prepared according to modified general procedure A using methylamine (5 equiv, 33 wt. % in EtOH, stirred at 30° C.) and (E/Z)-crotonyl chloride, affording 10 as a yellowish solid (23 mg, 15%, E:Z=3.7:1).

### Analytical Data for 10:

FTIR (diamond, anvil, solid) cm⁻¹:3270.87, 1591.03, 1546.36, 1436.05, 1396.65, 1263.00, 1192.48, 982.46, 967.32, 731.81, 699.25.

Signals present in both isomers: ¹H NMR (500 MHZ, CD₃OD) δ 8.14 (s, 1H), 7.60 (s, 1H), 7.47-7.35 (m, 2H), 6.89-6.84 (m, 1H) 5.75 (s, 1H), 2.86 (s, 3H) [protic N—H signals not observed].

Signals specific to (E)-9:6.94 (dq, J=13.9, 6.9 Hz, 1H), 6.15-6.08 (m, 1H), 1.92 (dd, J=6.9, 1.7 Hz, 3H).

Signals specific to (Z)-9:6.78 (dq, J=14.9, 6.8 Hz, 1H), 5.97-5.88 (m, 1H), 1.86 (dd, J=6.8, 1.7 Hz, 3H).

¹³C NMR (126 MHz, CD₃OD) δ 167.90, 165.50, 165.29, 157.58, 153.24, 141.39, 140.33, 139.14, 129.78, 124.94, 124.49, 116.52, 116.26, 112.60, 87.27, 26.76, 16.64, 16.44.

HRMS (ESI): Exact mass calcd for C₁₅H₁₇N₄O₂⁺ [M+H]⁺, 285.1346. Found 285.1347.

MEK7 IC₅₀>80 μM.

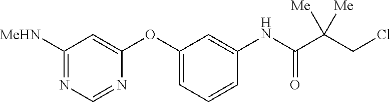 [SMILES: CNc1cc(Oc2cccc(NC(=O)C(C)(C)CCl)c2)ncn1]

3-chloro-2,2-dimethyl-N-(3-((6-(methylamino)pyrimidin-4-yl)oxy)phenyl) propenamide (11). Prepared according to modified general procedure A using methylamine (5 equiv, 33 wt. % in EtOH, stirred at 30° C.) and 3-chloro-2,2-dimethylpropanoyl chloride, isolated as a white solid (16 mg, 21%).

### Analytical Data for 11:

FTIR (diamond, anvil, solid) cm⁻¹:3299.52, 1590.12, 1538.25, 1486.59, 1432.53, 1394.61, 1255.28, 1191.62, 982.37, 731.54, 699.10.

¹H NMR (500 MHZ, CD₃OD) δ 8.13 (s, 1H), 7.52-7.42 (m, 2H), 7.38 (ap t, J=8.1 Hz, 1H), 6.90 (ap dd, J=8.1, 2.4 Hz, 1H), 5.73 (s, 1H), 3.76 (s, 2H), 2.84 (s, 3H), 1.37 (s, 6H) [protic N—H signals not observed].

¹³C NMR (126 MHz, CD₃OD) δ 174.73, 169.08, 165.51, 157.59, 153.08, 139.90, 129.62, 117.94, 116.77, 114.00, 87.04, 51.78, 44.96, 31.39, 22.34.

HRMS (ESI): Exact mass calcd for C₁₆H₂₀ClN₄O₂⁺ [M+H]⁺, 335.1269. Found 335.1270.

MEK7 IC₅₀>80 μM.

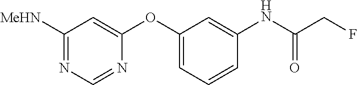 [SMILES: CNc1cc(Oc2cccc(NC(=O)CF)c2)ncn1]

2-fluoro-N-(3-((6-(methylamino)pyrimidin-4-yl)oxy)phenyl)acetamide (12). To a 0.1 M solution of 2-chloro-N-(3-((6-(methylamino)pyrimidin-4-yl)oxy)phenyl)acetamide (8) in DMSO was added CsF (10 equiv). The resulting solution was heated to 80° C. and was allowed to stir for a period of 12 hours. Upon cooling to room temperature, the reaction mixture was diluted with water (10 volumes) and extracted with Et₂O (3×1 volume). The combined organic extracts were dried over anhydrous Na₂SO₄, filtered through glass wool, and concentrated in vacuo. The product was purified by flash column chromatography (SiO2, 1:1 hexanes:acetone) to afford 12 (25 mg, 64%) as a pale yellow oil.

### Analytical Data for 12:

FTIR (diamond, anvil, solid) cm⁻¹:3281.55, 1684.66, 1597.18, 1556.75, 1489.00, 1441.31, 1259.63, 1193.64, 1046.74, 985.14.

¹H NMR (500 MHz, DMSO) δ 10.20 (s, 1H), 8.13 (s, 1H), 7.52-7.47 (m, 2H), 7.40-7.29 (m, 2H), 6.89-6.83 (m, 1H), 5.80 (s, 1H), 4.98 (d, J=46.9 Hz, 2H), 2.77 (s, 3H).

¹³C NMR (126 MHZ, DMSO) δ 166.67, 166.52, 166.10, 165.59, 158.52, 153.59, 139.78, 130.29, 117.17, 116.69, 113.14, 81.04, 79.61, 70.28, 28.00.

¹⁹F NMR (470 MHz, DMSO) δ −225.24.

HRMS (ESI): Exact mass calcd for C₁₃H₁₄FN₄O₂+ [M+H]⁺, 277.1095. Found 277.1100.

MEK7 IC₅₀: 7.8 μM [7.7±20, n=3; 7.8±5.0, n=3].

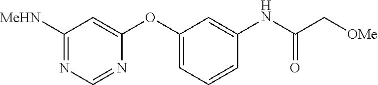 [SMILES: CNc1cc(Oc2cccc(NC(=O)COC)c2)ncn1]

2-methoxy-N-(3-((6-(methylamino)pyrimidin-4-yl)oxy)phenyl)acetamide (13). Prepared according to modified general procedure A using methylamine (5 equiv, 33 wt. % in EtOH, stirred at 30° C.) and methoxyacetyl chloride, isolated as an off-white solid (132 mg, 41%).

### Analytical Data for 13:

FTIR (diamond, anvil, solid) cm⁻¹:3292.99, 3246.01, 3101.16, 1618.86, 1528.42, 1441.91, 1314.17, 1297.79, 1144.36, 1020.26, 855.51, 720.78.

¹H NMR (500 MHZ, CD₃OD) δ 8.12 (s, 1H), 7.59-7.54 (m, 1H), 7.50-7.44 (m, 1H), 7.38 (ap t, J=8.1 Hz, 1H), 6.92-6.86 (m, 1H), 5.73 (s, 1H), 4.02 (s, 2H), 3.46 (s, 3H), 2.84 (s, 3H) [protic N—H signals not observed].

³C NMR (126 MHZ, CD₃OD) δ 170.87, 166.88, 158.95, 155.15, 154.57, 140.65, 131.14, 118.41, 118.14, 114.51, 88.47, 72.87, 59.59, 28.15.

HRMS (ESI): Exact mass calcd for C₁₄H₁₇N₄O₃+ [M+H]⁺, 289.1295. Found 289.1299.

MEK7 IC₅₀: >80 μM.

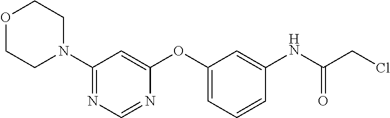 [SMILES: O=C(CCl)Nc1cccc(Oc2cc(N3CCOCC3)ncn2)c1]

2-chloro-N-(3-((6-morpholinopyrimidin-4-yl)oxy)phenyl)acetamide (14). Prepared according to general procedure A using morpholine and chloroacetyl chloride, isolated as a white solid (77 mg, 50%).

### Analytical Data for 14:

FTIR (diamond, anvil, solid) cm⁻¹:3068.16, 1593.46, 1557.52, 1487.86, 1440.22, 1333.49, 1202.91, 1157.54, 1010.08.

¹H NMR (500 MHz, DMSO) δ 10.43 (s, 1H), 8.22 (s, 1H), 7.47 (s, 1H), 7.44-7.32 (m, 2H), 6.88-6.82 (m, 1H), 6.33 (s, 1H), 4.25 (s, 2H), 3.66 (t, J=4.8 Hz, 4H), 3.57 (t, J=4.8 Hz, 4H).

¹³C NMR (126 MHz, DMSO) δ 170.00, 165.28, 164.56, 157.96, 153.64, 140.08, 130.28, 117.16, 116.14, 112.72, 87.31, 66.21, 44.51, 44.04.

HRMS (ESI): Exact mass calcd for C₁₆H₁₇ClN₄O₃Na⁺ [M+Na]⁺371.0881. Found 371.0881. MEK7 IC₅₀: 0.90 PM [1.3±3.4, n=3; 0.49±0.86, n=3].

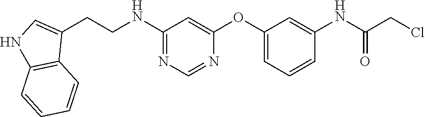 [SMILES: O=C(CCl)Nc1cccc(Oc2cc(NCCc3c[nH]c4ccccc34)ncn2)c1]

N-(3-((6-((2-(1H-indol-3-yl)ethyl)amino)pyrimidin-4-yl)oxy)phenyl)-2-chloroacetamide (15). Prepared according to general procedure A with tryptamine and chloroacetyl chloride, isolated as a white solid (71 mg, 39%).

### Analytical Data for 15:

FTIR (diamond, anvil, solid) cm⁻¹:3256.44, 3053.72, 1651.18, 1593.79, 1556.77, 1435.85, 1264.33, 1197.50, 1014.65, 731.35, 701.13.

¹H NMR (500 MHZ, CDCl₃) δ 8.32 (s, 1H), 8.21 (s, 1H), 8.16 (s, 1H), 7.57 (ap d, J=7.9 Hz, 1H), 7.48-7.38 (m, 1H), 7.38-7.29 (m, 3H), 7.23-7.16 (m, 1H), 7.15-7.07 (m, 1H), 7.02 (ap d, J=2.3 Hz, 1H), 6.94-6.89 (m, 1H), 5.71 (s, 1H), 5.15 (b s, 1H), 4.14 (s, 2H), 3.57 (s, 2H), 3.06 (t, J=6.7 Hz, 2H).

¹³C NMR (126 MHZ, CD₃OD) δ 169.01, 166.11, 164.94, 157.65, 153.30, 139.62, 136.74, 129.91, 127.32, 122.16, 122.08, 120.91, 118.22, 117.83, 116.86, 116.62, 112.72, 110.82, 87.39, 42.63, 41.67, 24.77.

HRMS (ESI): Exact mass calcd for C₂₂H₂₁ClN₅O₂⁺ [M+H]⁺422.1378. Found 422.1382.

MEK7 IC₅₀: 0.26 μM [0.30±0.092, n=3; 0.21±0.18 n=3].

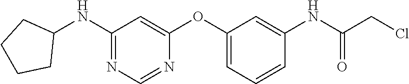 [SMILES: O=C(CCl)Nc1cccc(Oc2cc(NC3CCCC3)ncn2)c1]

2-chloro-N-(3-((6-(cyclopentylamino)pyrimidin-4-yl)oxy)phenyl)acetamide (16). Prepared according to general procedure A with cyclopentylamine and chloroacetyl chloride, isolated as a white solid (22 mg, 31%).

### Analytical Data for 16:

FTIR (diamond, anvil, solid) cm⁻¹:3339.69, 2952.97, 1594.04, 1489.59, 1442.81, 1250.88, 1156.43, 1013.57, 812.07, 688.92.

¹H NMR (500 MHz, DMSO) δ 10.47 (s, 1H), 8.13 (s, 1H), 7.47-7.33 (m, 4H), 6.89-6.84 (m, 1H), 5.76 (s, 1H), 4.29-4.24 (m, 3H), 1.88 (dq, J=12.9, 6.4 Hz, 2H), 1.64 (t, J=6.3 Hz, 2H), 1.57-1.48 (m, 2H), 1.43-1.40 (m, 2H).

¹³C NMR (126 MHz, DMSO) δ 169.13, 165.30, 164.64, 158.59, 153.64, 140.24, 130.50, 117.17, 116.25, 112.67, 87.74, 52.36, 44.02, 32.76, 23.82.

HRMS (ESI): Exact mass calcd for C₁₇H₂₀ClN₄O₂⁺ [M+H]⁺347.1269. Found 347.1271.

MEK7 IC₅₀: 0.27 μM [0.35±0.15, n=3; 0.19±0.19, n=3].

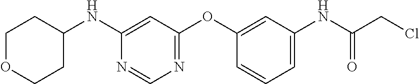 [SMILES: O=C(CCl)Nc1cccc(Oc2cc(NC3CCOCC3)ncn2)c1]

2-chloro-N-(3-((6-((tetrahydro-2H-pyran-4-yl)amino)pyrimidin-4-yl)oxy)phenyl)acetamide (17). Prepared according to general procedure A using tetrahydro-2H-pyran-4-amine and chloroacetyl chloride, isolated as an off-white solid (99 mg, 32%).

### Analytical Data for 17:

FTIR (diamond, anvil, solid) cm⁻¹:3266.20, 2957.00, 1591.97, 1438.51, 1419.02, 1260.29, 1184.21, 1138.54, 1011.39, 982.86, 732.24, 700.05.

¹H NMR (500 MHZ, DMSO) δ 10.44 (s, 1H), 8.15 (s, 1H), 7.45 (s, 1H), 7.43-7.35 (m, 3H), 6.87 (ap d, J=7.7 Hz, 1H), 5.78 (s, 1H), 4.25 (s, 2H), 4.00 (s, 1H), 3.83 (d, J=11.7 Hz, 2H), 3.38 ([obscured by water peak], 2H), 1.82 (d, J=12.7 Hz, 2H), 1.40 (d, J=11.9 Hz, 2H).

¹³C NMR (126 MHz, DMSO) δ 169.28, 165.31, 164.29, 158.67, 153.61, 140.24, 130.52, 117.19, 116.31, 112.70, 87.69, 66.31, 46.74, 44.01, 32.95.

HRMS (ESI): Exact mass calcd for C₁₇H₂₀ClN₄O₃+ [M+H]⁺, 363.1218. Found 363.1220.

MEK7 IC₅₀: 1.5 M [1.4±1.1, n=3; 1.6±1.1, n=3].

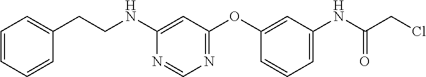 [SMILES: O=C(CCl)Nc1cccc(Oc2cc(NCCc3ccccc3)ncn2)c1]

2-chloro-N-(3-((6-phenethylpyrimidin-4-yl)oxy)phenyl)acetamide (18). Prepared according to general procedure A with 2-phenylethan-1-amine and chloroacetyl chloride, isolated as a white solid (76 mg, 61%).

### Analytical Data for 18:

FTIR (diamond, anvil, solid) cm⁻¹:3273.86, 3060.56, 1590.58, 1553.02, 1438.92, 1194.40, 1015.48, 983.66, 770.35, 731.94, 699.49.

¹H NMR (500 MHz, DMSO) δ 10.45 (s, 1H), 8.17 (s, 1H), 7.51-7.44 (m, 2H), 7.44-7.34 (m, 2H), 7.33-7.26 (m, 2H), 7.26-7.19 (m, 3H), 6.87 (s, 1H), 5.83 (s, 1H), 4.26 (s, 2H), 3.52 (s, 2H), 2.81 (t, J=6.5 Hz, 2H).

¹³C NMR (126 MHz, DMSO) δ 169.12, 165.30, 165.06, 158.66, 153.65, 140.22, 139.86, 130.48, 129.15, 128.80, 126.57, 117.15, 116.25, 112.68, 87.96, 44.02, 42.34, 35.41.

HRMS (ESI): Exact mass calcd for C₂₀H₂₀ClN₄O₂⁺ [M+H]⁺383.1269. Found 383.1274.

MEK7 IC₅₀: 1.2 μM [0.73±0.22, n=3; 1.7±11, n=3].

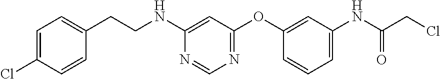 [SMILES: O=C(CCl)Nc1cccc(Oc2cc(NCCc3ccc(Cl)cc3)ncn2)c1]

2-chloro-N-(3-((6-((4-chlorophenethyl)amino)pyrimidin-4-yl)oxy)phenyl)acetamide (19). Prepared according to general procedure A with 2-(4-chlorophenyl) ethan-1-amine and chloroacetyl chloride, isolated as a white solid (91 mg, 43%).

### Analytical Data for 19:

FTIR (diamond, anvil, solid) cm⁻¹:3265.49, 2932.69, 1593.95, 1557.36, 1531.30, 1489.61, 1439.46, 1193.21, 1015.43, 824.61, 699.96.

¹H NMR (500 MHz, DMSO) δ 10.42 (s, 1H), 8.16 (s, 1H), 7.45 (s, 2H), 7.42-7.36 (m, 2H), 7.34 (ap d, J=8.4 Hz, 2H), 7.26 (ap d, J=8.4 Hz, 2H), 6.86 (ap d, J=7.3 Hz, 1H), 5.81 (s, 1H), 4.25 (s, 2H), 3.50 (s, 2H), 2.80 (d, J=6.5 Hz, 2H).

¹³C NMR (126 MHz, DMSO) δ 165.30, 165.04, 158.55, 153.63, 140.21, 138.93, 131.23, 131.08, 130.49, 129.58, 128.69, 117.15, 116.25, 112.67, 87.92, 44.02, 42.12, 34.68.

HRMS (ESI): Exact mass calcd for C₂₀H₁₉Cl₂N₄O₂+ [M+H]⁺417.0880. Found 417.0877.

MEK7 IC₅₀: 0.066 μM [0.053±0.26, n=3; 0.078±0.091, n=3].

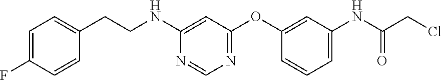 [SMILES: O=C(CCl)Nc1cccc(Oc2cc(NCCc3ccc(F)cc3)ncn2)c1]

2-chloro-N-(3-((6-((4-fluorophenethyl)amino)pyrimidin-4-yl)oxy)phenyl)acetamide (20). Prepared according to general procedure A with 2-(4-fluorophenyl) ethan-1-amine and chloroacetyl chloride, isolated as a white solid (57 mg, 44%).

### Analytical Data for 20:

FTIR (diamond, anvil, solid) cm⁻¹:3261.89, 2932.87, 1592.23, 1508.63, 1436.97, 1217.85, 1192.96, 1016.34, 823.80, 697.59.

¹H NMR (500 MHZ, CD₃OD) δ 8.13 (s, 1H), 7.53 (s, 1H), 7.46-7.37 (m, 2H), 7.20 (s, 2H), 6.99 (ap t, J=8.6 Hz, 2H), 6.90 (ap d, J=7.4 Hz, 1H), 5.71 (s, 1H), 4.18 (s, 2H), 3.55 (s, 2H), 2.83 (t, J=7.3 Hz, 2H) [protic N—H signals not observed].

¹³C NMR (126 MHz, CD₃OD) δ 166.12, 164.93, 162.58, 160.65, 157.69, 153.28, 152.80, 139.65, 135.06, 130.13 (d, J=7.9 Hz), 129.92, 116.75 (d, J=30.5 Hz), 114.66 (d, J=21.4 Hz), 112.76, 91.84, 42.62, 42.20, 34.20.

¹⁹F NMR (470 MHz, DMSO) δ −117.18.

HRMS (ESI): Exact mass calcd for C₂₀H₁₉ClFN₄O₂+ [M+H]⁺401.1175. Found 401.1175.

MEK7 IC₅₀: 1.2 μM [0.71±0.40, n=3; 1.6±1.8 n=3].

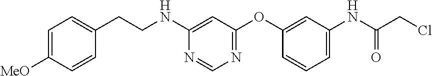 [SMILES: COc1ccc(CCNc2cc(Oc3cccc(NC(=O)CCl)c3)ncn2)cc1]

2-chloro-N-(3-((6-((4-methoxyphenethyl)amino)pyrimidin-4-yl)oxy)phenyl)acetamide (21). Prepared according to general procedure A with 2-(4-methoxyphenyl) ethan-1-amine and chloroacetyl chloride, isolated as a white solid (44 mg, 54%).

### Analytical Data for 21:

FTIR (diamond, anvil, solid) cm⁻¹:3266.42, 1591.95, 1513.80, 1439.00, 1259.84, 1233.60, 1192.41, 1140.07, 1024.29, 731.62, 698.01.

¹H NMR (500 MHZ, CDCl₃) δ 8.34 (s, 1H), 8.18 (s, 1H), 7.46 (ap t, J=2.0 Hz, 1H), 7.41-7.31 (m, 2H), 7.13-7.06 (m, 2H), 6.91 (ap dt, J=7.3, 2.2 Hz, 1H), 6.86-6.76 (m, 2H), 5.72 (s, 1H), 4.15 (s, 2H), 3.77 (s, 3H), 3.43 (s, 2H), 2.84 (t, J=7.1 Hz, 2H) [one protic N—H signal not observed].

¹³C NMR (126 MHZ, CDCl₃) δ 169.95, 163.99, 163.90, 158.41, 157.49, 153.22, 138.04, 130.21, 129.76, 127.82, 118.21, 116.94, 114.16, 113.47, 85.83, 55.32, 43.25, 42.88, 34.29.

HRMS (ESI): Exact mass calcd for C₂₁H₂₂ClN₄O₃+ [M+H]⁺413.1375. Found 413.1377.

MEK7 IC₅₀: 0.93 μM [1.2±0.33, n=3; 0.66±0.31, n=3].

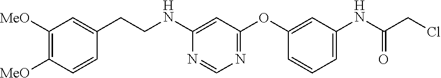 [SMILES: COc1ccc(CCNc2cc(Oc3cccc(NC(=O)CCl)c3)ncn2)cc1OC]

2-chloro-N-(3-((6-((3,4-dimethoxyphenethyl)amino)pyrimidin-4-yl)oxy)phenyl)acetamide (22). Prepared according to general procedure A with 2-(3,4-dimethoxyphenyl) ethan-1-amine and chloroacetyl chloride, isolated as a white solid (81 mg, 49%).

### Analytical Data for 22:

FTIR (diamond, anvil, solid) cm⁻¹:3257.62, 2936.66, 1652.12, 1594.52, 1558.11, 1514.76, 1438.94, 1260.55, 1234.56, 1156.50, 1025.30, 690.34.

¹H NMR (500 MHZ, CD₃OD) δ 8.12 (s, 1H), 7.52 (s, 1H), 7.48-7.36 (m, 2H), 6.89 (ap d, J=7.8 Hz, 1H), 6.86-6.78 (m, 2H), 6.77-6.70 (m, 1H), 5.70 (s, 1H), 4.17 (s, 2H), 3.78 (s, 6H), 3.54 (s, 2H), 2.78 (t, J=7.2 Hz, 2H) [protic N—H signals not observed].

¹³C NMR (126 MHZ, CD₃OD) δ 166.08, 164.93, 163.47, 157.67, 153.29, 148.95, 147.61, 139.64, 131.98, 129.91, 120.78, 116.86, 116.61, 112.72, 112.33, 111.66, 87.34, 55.08, 54.96, 42.64, 34.64.

HRMS (ESI): Exact mass calcd for C₂₂H₂₄ClN₄O₄+ [M+H]⁺443.1481. Found 443.1481.

MEK7 IC₅₀: 1.7 μM [1.9±0.55, n=3; 1.4±0.97 n=3].

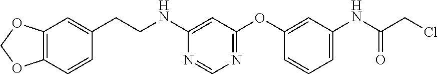 [SMILES: O=C(CCl)Nc1cccc(Oc2cc(NCCc3ccc4c(c3)OCO4)ncn2)c1]

N-(3-((6-((2-(benzo[d][1,3]dioxol-5-yl)ethyl)amino)pyrimidin-4-yl)oxy)phenyl)-2-chloroacetamide (23). Prepared according to general procedure A with 2-(benzo[d][1,3]dioxol-5-yl) ethan-1-amine and chloroacetyl chloride, isolated as a white solid (41 mg, 24%).

### Analytical Data for 23:

FTIR (diamond, anvil, solid) cm⁻¹:3338.06, 2926.72, 1593.90, 1488.86, 1441.66, 1247.83, 1157.44, 1013.91, 810.88, 688.84.

¹H NMR (500 MHz, CDCl₃) δ 8.31 (s, 1H), 8.24 (s, 1H), 7.48 (s, 1H), 7.42-7.33 (m, 2H), 6.95 (ap dt, J=7.6, 2.1 Hz, 1H), 6.76 (ap d, J=7.8 Hz, 1H), 6.71-6.62 (m, 2H), 5.94 (s, 2H), 5.75 (s, 1H), 5.06 (s, 1H), 4.18 (s, 2H), 3.48 (s, 2H), 2.83 (t, J=6.9 Hz, 2H).

¹³C NMR (126 MHZ, DMSO) δ 169.06, 165.30, 165.04, 161.45, 158.63, 153.65, 145.99, 140.22, 133.63, 130.47, 122.05, 117.14, 116.25, 112.67, 109.56, 108.58, 101.11, 87.96, 44.02, 42.50, 35.12.

HRMS (ESI): Exact mass calcd for C₂₁H₂₀ClN₄O₄+ [M+H]⁺427.1168. Found 427.1169.

MEK7 IC₅₀: 1.1 μM [1.4±0.81, n=3; 0.83±0.68, n=3].

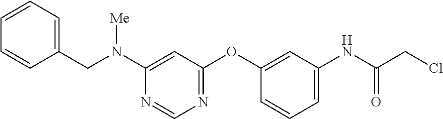 [SMILES: CN(Cc1ccccc1)c1cc(Oc2cccc(NC(=O)CCl)c2)ncn1]

N-(3-((6-(benzyl(methyl)amino)pyrimidin-4-yl)oxy)phenyl)-2-chloroacetamide (24). Prepared according to general procedure A using N-methylbenzylamine and chloroacetyl chloride, isolated as a white solid (54 mg, 41%).

### Analytical Data for 24:

FTIR (diamond, anvil, solid) cm⁻¹:3195.85, 3195.85, 1588.78, 1543.92, 1486.52, 1436.42, 1264.67, 1196.18, 1172.02, 1018.67, 731.13, 696.97.

¹H NMR (500 MHZ, CDCl₃) δ 8.49-8.45 (m, 1H), 8.27-8.23 (m, 1H), 7.38 (s, 1H), 7.31-7.23 (m, 3H), 7.23-7.18 (m, 2H), 7.17-7.09 (m, 2H), 6.87-6.80 (m, 1H), 5.85 (s, 1H), 4.75 (s, 2H), 4.03 (s, 2H), 2.96 (s, 3H).

¹³C NMR (126 MHz, CDCl₃) δ 169.94, 164.50, 164.10, 157.73, 153.55, 138.39, 137.21, 130.13, 128.84, 127.51, 127.20, 118.01, 116.87, 113.66, 86.12, 52.79, 43.01, 35.75.

HRMS (ESI): Exact mass calcd for C₂₀H₂₀ClN₄O₂⁺ [M+H]⁺383.1269. Found 383.1275.

MEK7 IC₅₀: 0.11 μM [0.14±0.087, n=3; 0.087±0.042, n=3].

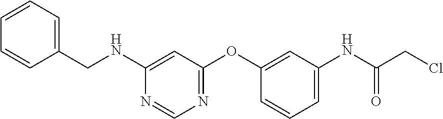 [SMILES: O=C(CCl)Nc1cccc(Oc2cc(NCc3ccccc3)ncn2)c1]

N-(3-((6-(benzylamino)pyrimidin-4-yl)oxy)phenyl)-2-chloroacetamide (25 DK2403). Prepared according to general procedure A using benzylamine and chloroacetyl chloride, isolated as a white solid (73 mg, 52%).

### Analytical Data for 25:

FTIR (diamond, anvil, solid) cm⁻¹:3262.47, 3029.05, 1594.33, 1487.64, 1258.20, 1027.03, 1002.40, 981.58, 819.88, 735.76.

¹H NMR (500 MHZ, DMSO) δ 10.44 (s, 1H), 8.16 (s, 1H), 7.91 (s, 1H), 7.41 (ap dd, J=24.1, 15.9 Hz, 3H), 7.34-7.27 (m, 4H), 7.24 (ap t, J=7.1 Hz, 1H), 6.89-6.82 (m, 1H), 5.88 (s, 1H), 4.52 (s, 2H), 4.26 (s, 2H).

¹³C NMR (126 MHz, DMSO) δ 169.26, 165.29, 165.13, 158.64, 153.58, 140.20, 140.05, 130.46, 128.83, 127.69, 127.32, 117.17, 116.27, 112.70, 87.96, 44.23, 44.02.

HRMS (ESI): Exact mass calcd for C₁₉H₁₈ClN₄O₂⁺ [M+H]⁺369.1113. Found 369.1118.

MEK7 IC₅₀: 10 nM [0.017±0.15 μM, n=3; IC₅₀<0.0014 μM, n=3].

Without preincubation, MEK7 IC₅₀: 93 nM [0.067±0.021 μM, n=3; 0.119 μM+0.039, n=3].

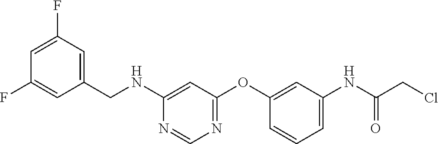 [SMILES: O=C(CCl)Nc1cccc(Oc2cc(NCc3cc(F)cc(F)c3)ncn2)c1]

2-chloro-N-(3-((6-((3,5-difluorobenzyl)amino)pyrimidin-4-yl)oxy)phenyl)acetamide (26). Prepared according to general procedure A using 3,5-difluorobenzylamine and chloroacetyl chloride, isolated as a white solid (34 mg, 28%).

### Analytical Data for 26:

FTIR (diamond, anvil, solid) cm⁻¹:3263.77, 3069.18, 2927.68, 1688.21, 1591.71, 1555.15, 1510.99, 1487.25, 1440.06, 1183.22, 1117.51, 988.42, 848.14, 690.49.

¹H NMR (500 MHz, DMSO) δ 10.42 (s, 1H), 8.16 (s, 1H), 7.95 (s, 1H), 7.45 (s, 1H), 7.43-7.33 (m, 2H), 7.09 (ap t, J=9.7 Hz, 1H), 7.00 (ap d, J=7.7 Hz, 2H), 6.87 (s, 1H), 5.91 (s, 1H), 4.53 (br s, 2H), 4.25 (s, 2H).

¹³C NMR (126 MHz, DMSO) δ 169.31, 165.30, 165.01, 163.84 (d, J=13.2 Hz), 161.89 (d, J=13.1 Hz), 158.63, 153.50, 145.29, 140.22, 130.48, 117.21, 116.34, 112.72, 110.59 (d, J=5.8 Hz), 110.43 (d, J=5.7 Hz), 102.70 (t, J=25.5 Hz) 88.21, 44.01, 43.51.

¹⁹F NMR (470 MHz, DMSO) δ −109.98.

HRMS (ESI): Exact mass calcd for C₁₉H₁₆ClF₂N₄O₂+ [M+H]⁺405.0924. Found 405.0930.

MEK7 IC₅₀: 0.074 μM [0.11±1.2 μM, n=3; 0.037±1.6 μM, n=3].

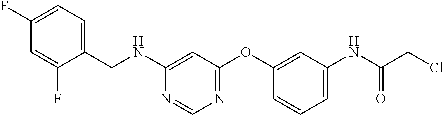 [SMILES: O=C(CCl)Nc1cccc(Oc2cc(NCc3ccc(F)cc3F)ncn2)c1]

2-chloro-N-(3-((6-((2,4-difluorobenzyl)amino)pyrimidin-4-yl)oxy)phenyl)acetamide (27). Prepared according to general procedure A using 2,4-difluorobenzylamine and chloroacetyl chloride, isolated as a white solid (22 mg, 19%).

### Analytical Data for 27:

FTIR (diamond, anvil, solid) cm⁻¹:3264.53, 3072.66, 1688.66, 1593.96, 1556.22, 1504.24, 1488.12, 1440.04, 1273.33, 1175.93, 979.79, 787.31.

¹H NMR (500 MHZ, DMSO) δ 10.44 (s, 1H), 8.17 (s, 1H), 7.88 (s, 1H), 7.45 (s, 1H), 7.42-7.34 (m, 3H), 7.22 (ap t, J=10.2 Hz, 1H), 7.05 (ap t, J=8.9 Hz, 1H), 6.89-6.84 (m, 1H), 5.89 (s, 1H), 4.51 (s, 2H), 4.25 (s, 2H).

¹³C NMR (126 MHz, DMSO) δ 169.23, 165.31, 164.90, 162.23 (dd, J=160.7, 12.2 Hz), 160.28 (dd, J=162.7, 12.4 Hz), 158.64, 153.50, 140.23, 134.67, 131.18, 130.52, 117.21, 116.33, 112.69, 111.80 (d, J=21.2 Hz), 104.22 (t, J=25.8 Hz), 87.99, 44.02, 37.73.

¹⁹F NMR (470 MHz, DMSO) δ −112.05, −114.48.

HRMS (ESI): Exact mass calcd for C₁₉H₁₆ClF₂N₄O₂+ [M+H]⁺405.0924. Found 405.0928.

MEK7 IC₅₀: 0.36 μM [0.14±0.65], n=3; 0.58±0.20, n=3].

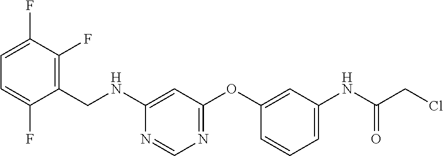 [SMILES: O=C(CCl)Nc1cccc(Oc2cc(NCc3c(F)ccc(F)c3F)ncn2)c1]

2-chloro-N-(3-((6-((2,3,6-trifluorobenzyl)amino)pyrimidin-4-yl)oxy)phenyl)acetamide (28). Prepared according to general procedure A using (2,3,6-trifluorophenyl) methanamine and chloroacetyl chloride, isolated as a white solid (61 mg, 33%).

### Analytical Data for 28:

FTIR (diamond, anvil, solid) cm⁻¹:3254.75, 3058.39, 1592.32, 1494.24, 1244.30, 1216.36, 1183.06, 1015.45, 810.65, 733.18, 698.70.

¹H NMR (500 MHZ, DMSO) δ 10.51 (s, 1H), 8.20 (s, 1H), 7.87 (s, 1H), 7.47-7.35 (m, 4H), 7.16-7.07 (m, 1H), 6.86 (ap ddd, J=7.9, 2.4, 1.2 Hz, 1H), 5.86 (s, 1H), 4.58 (s, 2H), 4.25 (s, 2H).

¹³C NMR (126 MHz, DMSO) δ 169.23, 165.36, 164.57, 158.57, 157.11 (dd, J=243.6, 5.3 Hz), 153.42, 149.95-147.84 (m), 147.83-145.84 (m), 140.24, 130.57, 117.23, 117.11 (d, J=9.7 Hz), 116.41, 112.69, 111.84 (dd, J=24.8, 2.5 Hz), 87.97, 43.99, 32.89.

¹⁹F NMR (470 MHz, DMSO) δ −119.21, −138.15, −143.09.

HRMS (ESI): Exact mass calcd for C₁₉H₁₅ClF₃N₄O₂+ [M+H]⁺423.0830. Found 423.0836.

MEK7 IC₅₀: 0.11 μM [0.88±0.029, n=3; 0.14±0.044, n=3].

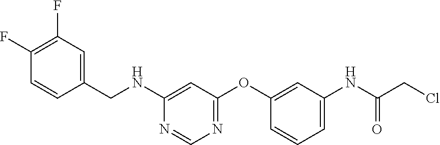 [SMILES: O=C(CCl)Nc1cccc(Oc2cc(NCc3ccc(F)c(F)c3)ncn2)c1]

2-chloro-N-(3-((6-((3,4-difluorobenzyl)amino)pyrimidin-4-yl)oxy)phenyl)acetamide (29). Prepared according to general procedure A with (3,4-difluorophenyl) methanamine and chloroacetyl chloride, isolated as a white solid (32 mg, 21%).

### Analytical Data for 29:

FTIR (diamond, anvil, solid) cm⁻¹:3262.89, 3065.28, 1591.97, 1516.88, 1434.51, 1275.57, 1184.95, 1002.23, 778.28, 733.57, 698.70.

¹H NMR (500 MHz, DMSO) δ 10.45 (s, 1H), 8.16 (s, 1H), 7.95 (s, 1H), 7.46 (s, 1H), 7.44-7.28 (m, 4H), 7.19-7.11 (m, 1H), 6.90-6.83 (m, 1H), 5.90 (s, 1H), 4.50 (s, 2H), 4.32-4.24 (m, 2H).

¹³C NMR (126 MHz, DMSO) δ 169.31, 165.30, 164.98, 158.61, 153.52, 150.26 (dd, J=114.6, 12.6 Hz), 148.31 (dd, J=113.3, 12.6 Hz), 140.23, 137.92, 130.46, 124.25 (dd, J=6.6, 3.4 Hz), 117.81 (d, J=17.0 Hz), 117.20, 116.58 (d, J=17.2 Hz), 116.32, 112.72, 88.01, 44.01, 43.21.

¹⁹F NMR (470 MHz, DMSO) δ −138.85, −141.51.

HRMS (ESI): Exact mass calcd for C₁₉H₁₆ClF₂N₄O₂+ [M+H]⁺405.0924. Found 405.0928.

MEK7 IC₅₀: 0.042 μM [0.042±0.036, n=3; 0.042±0.058, n=3].

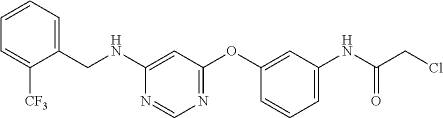 [SMILES: O=C(CCl)Nc1cccc(Oc2cc(NCc3ccccc3C(F)(F)F)ncn2)c1]

2-chloro-N-(3-((6-((2-(trifluoromethyl)benzyl)amino)pyrimidin-4-yl)oxy)phenyl)acetamide (30). Prepared according to general procedure A using (2-(trifluoromethyl)phenyl) methanamine and chloroacetyl chloride, isolated as a white solid (21 mg, 18%).

### Analytical Data for 30:

FTIR (diamond, anvil, solid) cm⁻¹:3255.28, 3013.33, 1592.26, 1555.54, 1486.66, 1436.71, 1253.39, 1212.54, 1171.75, 1023.80, 731.72, 694.88.

¹H NMR (500 MHz, DMSO) δ 10.46 (s, 1H), 8.17 (s, 1H), 7.97 (s, 1H), 7.72 (ap d, J=7.8 Hz, 1H), 7.64 (ap t, J=7.6 Hz, 1H), 7.53-7.44 (m, 3H), 7.43-7.34 (m, 2H), 6.88 (ap d, J=13.8 Hz, 1H), 5.98 (s, 1H), 4.73 (s, 2H), 4.26 (s, 2H).

¹³C NMR (126 MHz, DMSO) δ 169.26, 165.32, 164.98, 158.74, 153.43, 140.26, 133.22, 130.55, 129.04, 128.61 (d, J=262.2 Hz), 127.82, 126.68 (d, J=30.1 Hz), 126.30 (d, J=5.9 Hz), 124.96 (q, J=274.0 Hz), 117.24, 116.37, 112.70, 88.08, 44.04, 37.35.

¹⁹F NMR (470 MHz, DMSO) δ −58.92.

HRMS (ESI): Exact mass calcd for C₂₀H₁₇ClF₃N₄O₂+ [M+H]⁺437.0987. Found 437.0984.

MEK7 IC₅₀: 1.2 μM [1.1±0.48, n=3; 1.3±0.55, n=3].

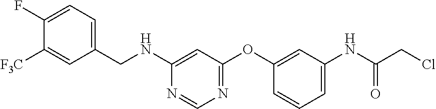 [SMILES: O=C(CCl)Nc1cccc(Oc2cc(NCc3ccc(F)c(C(F)(F)F)c3)ncn2)c1]

2-chloro-N-(3-((6-((4-fluoro-3-(trifluoromethyl)benzyl)amino)pyrimidin-4-yl)oxy)phenyl)acetamide (31). Prepared according to general procedure A using (4-fluoro-3-(trifluoromethyl)phenyl) methanamine and chloroacetyl chloride, isolated as a white solid (39 mg, 36%).

### Analytical Data for 31:

FTIR (diamond, anvil, solid) cm⁻¹:3392.39, 1651.24, 1600.14, 1436.55, 1316.25, 1236.99, 1129.17, 1020.83, 952.27, 703.73, 668.54.

¹H NMR (500 MHz, DMSO) δ 10.44 (s, 1H), 8.17 (s, 1H), 7.98 (s, 1H), 7.75-7.64 (m, 2H), 7.51-7.44 (m, 2H), 7.38 (ap d, J=11.2 Hz, 2H), 6.87 (ap d, J=7.3 Hz, 1H), 5.92 (s, 1H), 4.57 (s, 2H), 4.26 (s, 2H).

¹³C NMR (126 MHz, DMSO) δ 169.31, 165.30, 164.94, 158.65, 158.29 (d, J=252.66 Hz), 153.48, 140.22, 137.28, 134.42 (d, J=8.7 Hz), 130.48, 126.34 (d, J=4.8 Hz), 123.14 (d, J=272.3 Hz), 117.64 (d, J=20.4 Hz), 117.20, 116.77 (q, J=12.6 Hz), 116.33, 112.72, 88.02, 44.01, 43.11.

¹⁹F NMR (470 MHz, DMSO) δ −59.99, −118.90.

HRMS (ESI): Exact mass calcd for C₂₀H₁₅ClF₄N₄NaO₂⁺ [M+Na]⁺477.0712. Found 477.0715. MEK7 IC₅₀: 3.0 μM [4.2±2.5, n=3; 1.8±1.2, n=3].

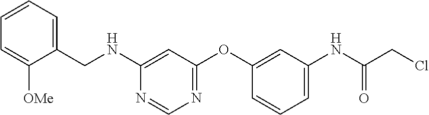 [SMILES: COc1ccccc1CNc1cc(Oc2cccc(NC(=O)CCl)c2)ncn1]

2-chloro-N-(3-((6-((2-methoxybenzyl)amino)pyrimidin-4-yl)oxy)phenyl)acetamide (32). Prepared according to general procedure A using (2-methoxyphenyl) methanamine and chloroacetyl chloride, isolated as a white solid (55 mg, 28%).

### Analytical Data for 32:

FTIR (diamond, anvil, solid) cm⁻¹:3374.85, 1651.71, 1598.62, 1558.77, 1489.87, 1437.03, 1244.19, 1218.19, 1019.06, 951.28, 756.87, 703.48.

¹H NMR (500 MHZ, DMSO) δ 10.43 (s, 1H), 8.14 (s, 1H), 7.71 (s, 1H), 7.45-7.34 (m, 3H), 7.24 (ap t, J=7.8 Hz, 1H), 7.18 (ap d, J=7.4 Hz, 1H), 6.99 (ap d, J=8.2 Hz, 1H), 6.93-6.84 (m, 2H), 5.91 (s, 1H), 4.48 (s, 2H), 4.26 (s, 2H), 3.80 (s, 3H).

¹³C NMR (126 MHz, DMSO) δ 169.04, 165.36, 165.15, 158.67, 157.20, 153.52, 140.16, 130.58, 128.68, 128.22, 127.18, 120.62, 117.23, 116.31, 112.65, 110.96, 88.00, 55.74, 43.98, 28.56.

HRMS (ESI): Exact mass calcd for C₂₀H₁₉ClN₄O₃Na⁺ [M+Na]⁺421.1038. Found 421.1039. MEK7 IC₅₀: 0.80 μM [0.59±0.15, n=3; 1.0±0.22, n=3].

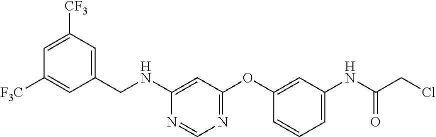 [SMILES: O=C(CCl)Nc1cccc(Oc2cc(NCc3cc(C(F)(F)F)cc(C(F)(F)F)c3)ncn2)c1]

N-(3-((6-((3,5-bis(trifluoromethyl)benzyl)amino)pyrimidin-4-yl)oxy)phenyl)-2-chloroacetamide (33). Prepared according to general procedure A with (3,5-bis-(trifluoromethyl)phenyl) methanamine and chloroacetyl chloride, isolated as a white solid (74 mg, 41%).

### Analytical Data for 33:

FTIR (diamond, anvil, solid) cm⁻¹:3255.56, 3066.66, 1594.99, 1437.82, 1277.61, 1171.99, 1129.49, 1024.69, 705.01, 682.10.

¹H NMR (500 MHz, DMSO) δ 10.46 (s, 1H), 8.18 (s, 1H), 8.10-7.95 (m, 4H), 7.47 (s, 1H), 7.44-7.34 (m, 2H), 6.88 (s, 1H), 5.97 (s, 1H), 4.71 (s, 2H), 4.26 (s, 2H).

¹³C NMR (126 MHz, DMSO) δ 169.29, 165.31, 164.92, 158.65, 153.42, 143.82, 140.23, 130.64 (q, J=32.7 Hz), 130.51, 128.47, 123.72 (q, J=272.7 Hz), 121.15, 117.21, 116.35, 112.73, 88.20, 44.02, 43.41.

¹⁹F NMR (470 MHz, DMSO) δ −61.34.

HRMS (ESI): Exact mass calcd for C₂₁H₁₆ClF₆N₄O₂+ [M+H]⁺505.0860. Found 505.0862.

MEK7 IC₅₀: 6.3μ M [4.0±4.4, n=3; 8.5±9.8, n=3].

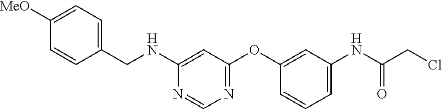 [SMILES: COc1ccc(CNc2cc(Oc3cccc(NC(=O)CCl)c3)ncn2)cc1]

2-chloro-N-(3-((6-((4-methoxybenzyl)amino)pyrimidin-4-yl)oxy)phenyl)acetamide (34). Prepared according to general procedure A with (4-methoxyphenyl) methanamine and chloroacetyl chloride, isolated as a white solid (90 mg, 55%).

### Analytical Data for 34:

FTIR (diamond, anvil, solid) cm⁻¹:3259.11, 3004.80, 1593.78, 1557.36, 1511.88, 1438.00, 1245.92, 1176.26, 1027.85, 819.54, 689.76.

¹H NMR (500 MHZ, CDCl₃) δ 8.38 (s, 1H), 8.23 (s, 1H), 7.44 (s, 1H), 7.40-7.29 (m, 2H), 7.26-7.20 (m, 2H), 6.95-6.83 (m, 3H), 5.76 (s, 1H), 5.61 (b s, 1H), 4.39 (s, 2H), 4.15 (s, 2H), 3.80 (s, 3H).

¹³C NMR (126 MHz, CDCl₃) δ 169.84, 164.46, 163.87, 159.16, 158.13, 153.31, 138.06, 130.15, 129.32, 128.77, 118.18, 116.82, 114.69, 114.24, 113.47, 55.36, 45.32, 42.89.

HRMS (ESI): Exact mass calcd for C₂₀H₂₀ClN₄O₃+ [M+H]⁺399.1218. Found 399.1223.

MEK7 IC₅₀: 0.30 PM [0.56±0.80, n=3; 0.041±0.054 n=3].

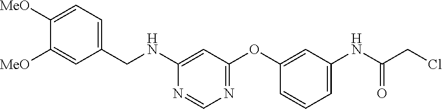 [SMILES: COc1ccc(CNc2cc(Oc3cccc(NC(=O)CCl)c3)ncn2)cc1OC]

2-chloro-N-(3-((6-((3,4-dimethoxybenzyl)amino)pyrimidin-4-yl)oxy)phenyl)acetamide (35). Prepared according to general procedure A with (3,4-dimethoxyphenyl)methanamine and chloroacetyl chloride, isolated as a white solid (67 mg, 51%).

### Analytical Data for 35:

FTIR (diamond, anvil, solid) cm⁻¹:3257.95, 3005.25, 1593.57, 1514.29, 1439.12, 1260.40, 1219.17, 1184.24, 1139.86, 1024.49, 805.47.

¹H NMR (500 MHZ, CDCl₃) δ 8.40 (s, 1H), 8.23 (s, 1H), 7.46 (ap t, J=2.1 Hz, 1H), 7.38-7.29 (m, 2H), 6.93-6.88 (m, 1H), 6.87-6.80 (m, 3H), 5.77 (ap d, J=0.9 Hz, 1H), 5.55 (b s, 1H), 4.39 (s, 2H), 4.15 (s, 2H), 3.87 (s, 3H), 3.86 (s, 3H).

¹³C NMR (126 MHz, CDCl₃) δ 169.83, 164.53, 163.92, 158.16, 153.28, 149.25, 148.56, 138.09, 130.14, 129.80, 119.69, 118.13, 116.83, 113.46, 111.23, 110.50, 86.50, 55.97, 55.92, 45.70, 42.90.

HRMS (ESI): Exact mass calcd for C₂₁H₂₂ClN₄O₄+ [M+H]⁺429.1324. Found 429.1327.

MEK7 IC₅₀: 0.26 μM [0.39±0.73, n=3; 0.12±0.38 n=3].

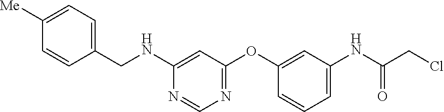 [SMILES: Cc1ccc(CNc2cc(Oc3cccc(NC(=O)CCl)c3)ncn2)cc1]

2-chloro-N-(3-((6-((4-methylbenzyl)amino)pyrimidin-4-yl)oxy)phenyl)acetamide (36). Prepared according to general procedure A with p-tolylmethanamine and chloroacetyl chloride, isolated as a white solid (31 mg, 47%).

### Analytical Data for 36:

FTIR (diamond, anvil, solid) cm⁻¹:3256.36, 2963.60, 1594.11, 1558.30, 1438.12, 1260.10, 1210.20, 1090.51, 1023.20, 799.08, 700.71.

¹H NMR (500 MHZ, DMSO) δ 10.42 (s, 1H), 8.14 (s, 1H), 7.85 (s, 1H), 7.45-7.35 (m, 3H), 7.20-7.15 (m, 2H), 7.15-7.10 (m, 2H), 6.86 (s, 1H), 5.85 (s, 1H), 4.46 (s, 2H), 4.25 (s, 2H), 2.27 (s, 3H).

¹³C NMR (126 MHz, DMSO) δ 169.24, 165.08, 158.60, 153.58, 152.95, 140.31, 140.19, 136.37, 130.46, 129.37, 127.68, 117.17, 116.25, 112.68, 87.92, 44.02, 21.14 [tolyl carbon obscured by DMSO].

HRMS (ESI): Exact mass calcd for C₂₀H₂₀ClN₄O₂⁺ [M+H]⁺383.1269. Found 383.1273.

MEK7 IC₅₀: 0.36 UM [0.42±0.32, n=3; 0.30±0.15 n=3].

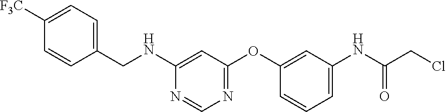 [SMILES: O=C(CCl)Nc1cccc(Oc2cc(NCc3ccc(C(F)(F)F)cc3)ncn2)c1]

2-chloro-N-(3-((6-((4-(trifluoromethyl)benzyl)amino)pyrimidin-4-yl)oxy)phenyl)acetamide (37). Prepared according to general procedure A with (4-(trifluoromethyl)phenyl) methanamine and chloroacetyl chloride, isolated as a white solid (90 mg, 55%).

### Analytical Data for 37:

FTIR (diamond, anvil, solid) cm⁻¹:3264.92, 3068.54, 1594.87, 1557.75, 1419.81, 1324.47, 1164.13, 1120.55, 1065.57, 1017.83, 817.17, 734.73, 700.32.

¹H NMR (500 MHz, DMSO) δ 10.43 (s, 1H), 8.17 (s, 1H), 8.02 (s, 1H), 7.70 (ap d, J=8.3 Hz, 2H), 7.51 (ap d, J=8.4 Hz, 2H), 7.46 (s, 1H), 7.43-7.34 (m, 2H), 6.87 (s, 1H), 5.91 (s, 1H), 4.61 (s, 2H), 4.26 (s, 2H).

¹³C NMR (126 MHz, DMSO) δ 169.21, 165.04, 161.76, 158.54, 153.48, 144.43, 140.23, 130.49, 128.24, 127.17 (q, J=271.0 Hz), 125.70, 123.73, 117.18, 116.36, 112.71, 87.99, 44.01, 40.82.

¹⁹F NMR (470 MHz, DMSO) δ −60.77.

HRMS (ESI): Exact mass calcd for C₂₀H₁₇ClF₃N₄O₂+ [M+H]⁺437.0987. Found 437.0988.

MEK7 IC₅₀: 0.41 μM [0.20±0.41, n=3; 0.64±0.27 n=3].

We claim:

1. A compound having a formula I, or a salt or hydrate thereof: 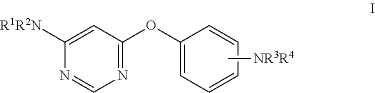
wherein
R¹ is hydrogen or alkyl;
R² is selected from the group consisting of alkyl, cycloalkyl, heterocycloalkyl, -alkylene-heteroaryl, -alkylene-aryl, wherein aryl is optionally substituted with one or more substituents selected from the group consisting of halo, alkyl, haloalkyl, and alkoxy;
or —NR¹R² together form a heterocycloalkyl;
R³ is hydrogen or alkyl;
R⁴ is —C(O)R⁵; and
R⁵ is alkenyl or alkyl optionally substituted with one or more substituents selected from halo and alkoxy.

2. The compound of claim 1 having a formula I(a) or I(b):

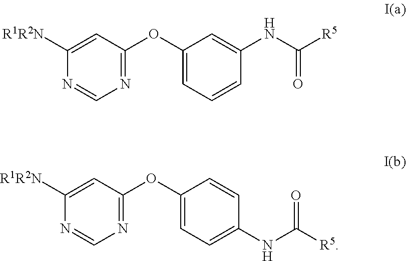

3. The compound of claim 2, wherein
the compound has the formula I(a);
R¹ is hydrogen;
R² is alkyl or —(CH₂)ₙ-aryl, wherein aryl is optionally substituted with one or more halo;
R⁵ is chloromethyl; and
n is an integer of 1-6.

4. The compound of claim 3, wherein the compound is

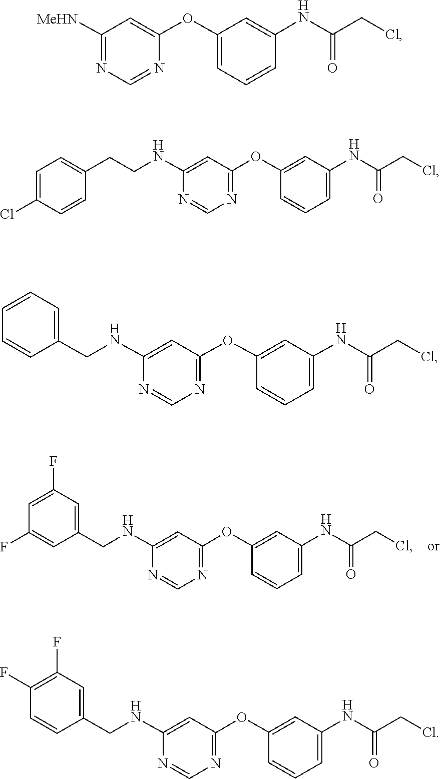

5. The compound of claim 3, wherein the compound is

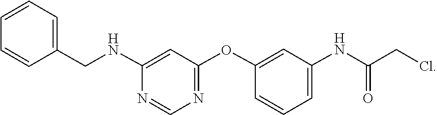 [SMILES: O=C(CCl)Nc1cccc(Oc2cc(NCc3ccccc3)ncn2)c1]

6. The compound of claim 2, wherein
the compound has the formula I(a);
R¹ is hydrogen or methyl;
R² is selected from the group consisting of alkyl, C₃-C₈-cycloalkyl, —(CH₂)ₙ-aryl, and —(CH₂)ₙ-heteroaryl, wherein aryl is optionally substituted with one or more substituents selected from the group consisting of alkyl, alkoxy, halo, and haloalkyl;
or —NR¹R² together form a heterocycloalkyl containing 1-3 heteroatoms selected from N, O, and S;
R⁵ is vinyl or chloromethyl; and
n is an integer of 1-6.

7. The compound of claim 6, wherein the compound is

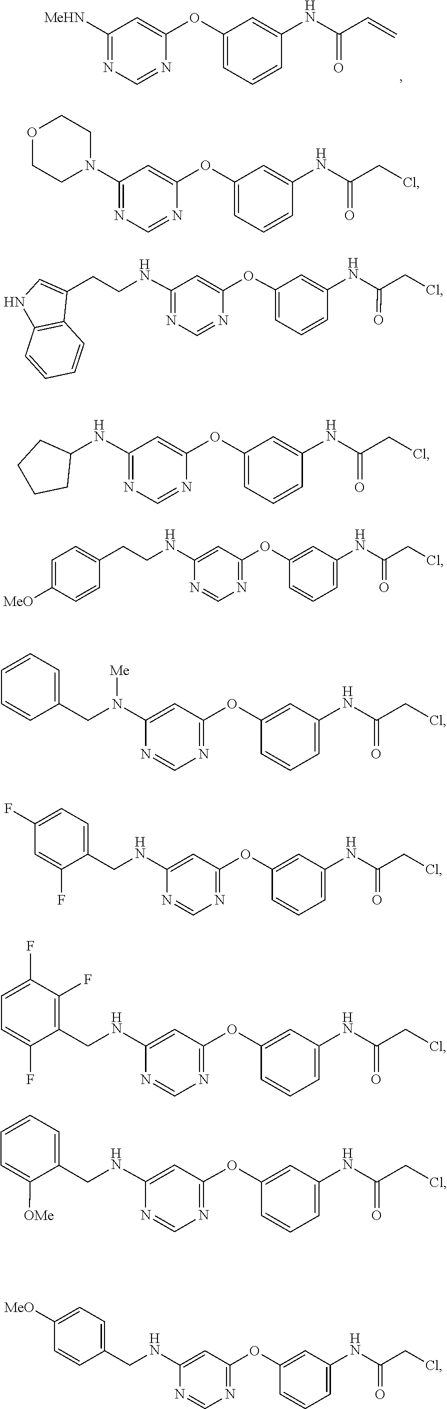

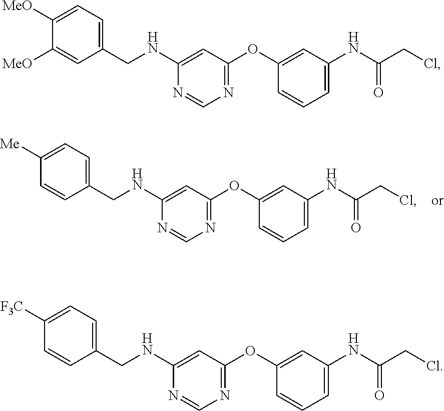

8. The compound of claim 2, wherein
the compound has the formula I(a);
R¹ is hydrogen;
R² is selected from the group consisting of alkyl, heterocycloalkyl containing 1-3 heteroatoms selected from N, O, and S, and —(CH₂)ₙ-aryl, wherein aryl is optionally substituted with one or more substituents selected from the group consisting of halo, alkoxy, and haloalkyl;
R⁵ is fluoromethyl or chloromethyl; and
n is integer of 1-6.

9. The compound of claim 8, wherein the compound is

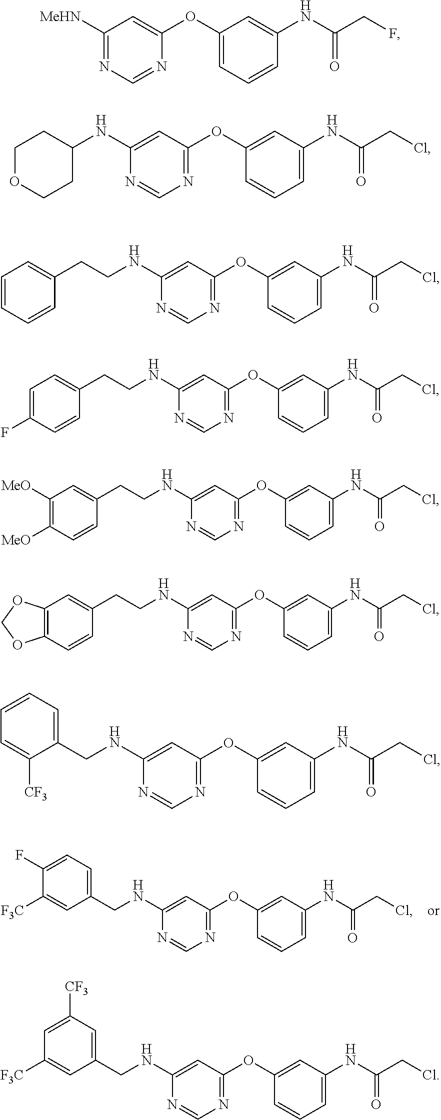

10. The compound of claim 2, wherein
the compound has the formula I(b);
R¹ is hydrogen;
R² is alkyl; and
R⁵ is vinyl or chloromethyl.

11. The compound of claim 10, wherein the compound is

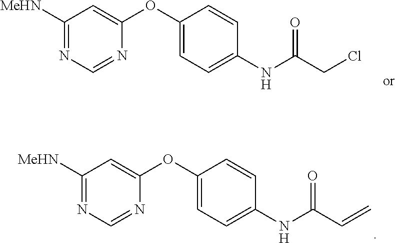 [SMILES: CNc1cc(Oc2ccc(NC(=O)CCl)cc2)ncn1 ; C=CC(=O)Nc1ccc(Oc2cc(NC)ncn2)cc1]

12. The compound of claim 2, wherein
the compound has the formula I(a);
R¹ is hydrogen;
R² is alkyl; and
R⁵ is propenyl or alkyl substituted with chloro or methoxy.

13. The compound of claim 12, wherein the compound is

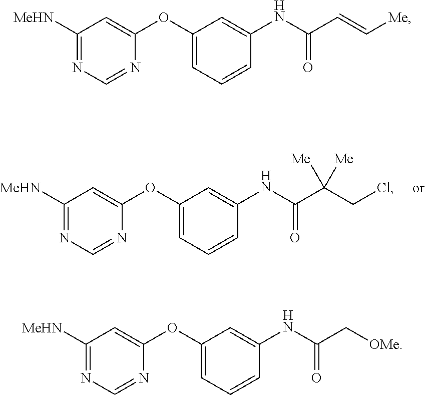

14. The compound of claim 1, wherein the compound is a selective MEK7 inhibitor.

15. A pharmaceutical composition comprising an effective amount of the compound of claim 1 and a pharmaceutical carrier, excipient, or diluent.

16. A method for treating a disease or disorder associated with mitogen-activated protein kinase 7 (MEK7) activity in a subject in need thereof, the method comprising administering to the subject the compound of claim 1.

17. The method of claim 16, wherein the disease or disorder is a cell proliferative disease or disorder.

18. The method of claim 17, wherein the cell proliferative disease or disorder is cancer.

19. The method of claim 18, wherein the cancer is acute lymphoblastic leukemia.

20. The method of claim 19, wherein the acute lymphoblastic leukemia is pediatric T-cell acute lymphoblastic leukemia.


SEQUENCE LISTING (2 sequences)

SEQ ID NO: 1 - protein, 21 aa, synthetic construct
  ACT_SITE 16: note = Cys218
ITNTDVFIAMELMGTCAEKLK

SEQ ID NO: 2 - protein, 16 aa, synthetic construct
LATGQFPYKNCKTDFE